(19) United States
(12) Patent Application Publication
(10) Pub. No.: US 2025/0134828 A1
(43) Pub. Date: May 1, 2025

(54) MOLECULAR SOLID SUSPENSIONS FOR TRANSDERMAL DRUG DELIVERY

(71) Applicant: Starton Therapeutics, Inc., Paramus, NJ (US)

(72) Inventors: Rod L. Hartwig, Hamburg, NJ (US); Arturo Serrano-Batista, Riverdale, NJ (US)

(21) Appl. No.: 18/933,332

(22) Filed: Oct. 31, 2024

Related U.S. Application Data
(60) Provisional application No. 63/594,986, filed on Nov. 1, 2023.

(51) Int. Cl. A61K 9/70; A61K 31/454; A61K 31/519; A61K 31/5513; A61K 31/573; A61K 47/10

(52) U.S. Cl. CPC A61K 9/7053; A61K 31/454; A61K 31/519; A61K 31/5513; A61K 31/573; A61K 47/10

(57) ABSTRACT

Transdermal drug delivery systems and methods of fabricating such systems are provided. The active pharmaceutical ingredient (API) can have a water solubility of less than about 10 milligrams per milliliter, a melting point greater than about 120° C., and a log P value ranging from about −2 to about 8. More particularly, the present disclosure is directed to improving the permeation of such compounds through the skin by forming a molecular solid suspension of the drug (API) on a carrier suspended within a drug-in-adhesive layer.

### RELATED APPLICATIONS

The present application claims priority to U.S. Provisional Patent Application No. 63/594,986, filed on Nov. 1, 2023, the entire contents of which are incorporated herein by reference.

### TECHNICAL FIELD

The present disclosure is directed to transdermal drug delivery systems for active pharmaceutical ingredients (APIs) exhibiting high melting points and low water solubility to improve the permeation of such APIs through the skin.

### BACKGROUND

The Food and Drug Administration (FDA) has established a system known as the Biopharmaceuticals Classification System (BCS), which is used to classify drug substances based on their permeability and solubility. Drug substances in category I have high permeability and high solubility, drug substances in category II have high permeability and low solubility, drug substances in category Ill have low permeability and high solubility, and drug substances in category IV have low permeability and low solubility. Transdermal delivery of solubilized APIs is well-known to those skilled in the art. Specifically, those APIs in most solubilized platforms are in the Class I or Class III categories of the FDA's BCS, such as nicotine, scopolamine, and methylphenidate, which have relatively low melting points and high water solubility. Meanwhile, many APIs in development or recently launched in the past few years have been assigned to BCS categories II and IV, which exhibit low solubility as defined by the FDA. Of commercially viable APIs made into transdermal drug delivery systems, applicable APIs might include buprenorphine, clonidine, estradiol, ethinyl estradiol, and norelgestromin, which would fit into the BCS category II, which includes drugs that exhibit low solubility but high permeability. Additionally, these drugs have relatively high melting points. These are incorporated into their respective commercially viable products in a solubilized state. Meanwhile, BCS category IV drugs exhibit low solubility and low permeability. The biopharmaceutical classifications are provided for reference in Table 1:

TABLE 1
Derivation of USP/NF solubility chart
to illustrate concentration types

| BCS Classification | Solubility | Permeability |
|---|---|---|
| I | High | High |
| II | Low | High |
| III | High | Low |
| IV | Low | Low |

Table 2 provides the basic physicochemical characteristics and BCS classifications of commercially viable transdermally delivered active pharmaceutical ingredients.

TABLE 2
Commercially Viable Transdermal
Active Pharmaceutical Ingredients

| | | Melt Point | Water Sol, | BCS |
|---|---|---|---|---|
| API | Mol. Wt. | ° C. | mg/mL | Class |
| Ethinyl Estradiol | 296.41 | 182 | ~0.1 | IV |
| Estradiol | 272.18 | 173 | ~0.1 | IV |
| Fentanyl | 336.48 | 82 | ~10 | II |
| Buprenorphine | 467.64 | 260 | ~10 | II |
| Granisetron | 314.42 | 219 | ~40 | II |
| Nicotine | 162.20 | −79 | ~60 | I |
| Methylphenidate | 233.31 | 74 | ~10 | II |
| Clonidine | 230.09 | 130 | ~1 | II |
| Scopolamine | 303.36 | 59 | ~100 | I |

To clarify the meaning of low solubility in the present application (sparingly soluble to practically insoluble in water), Table 3 below can be used for reference:

TABLE 3
Derivation of USP/NF solubility chart to illustrate concentration types

| Term | Parts solute | Parts solvent | Ratio | % | ppm | mg/g |
|---|---|---|---|---|---|---|
| Very Soluble | 1 | 1 | 0.5000 | 50 | 500,000 | 500 |
| Freely Soluble | 1 | 10 | 0.1000 | 10 | 100,000 | 100 |
| Soluble | 1 | 30 | 0.0333 | 3.33 | 33,333 | 33.33 |
| Sparingly Soluble | 1 | 100 | 0.0100 | 1 | 10,000 | 10 |
| Slightly Soluble | 1 | 1,000 | 0.0010 | 0.1 | 1,000 | 1 |
| Very Slightly Soluble | 1 | 10,000 | 0.0001 | 0.01 | 100 | 0.10 |
| Practically Insoluble | 1 | 100,000 | 0.00001 | 0.001 | 10 | 0.01 |

Such low solubility APIs can have a solubility of less than about 10 mg/g or 10 mg/mL. APIs of the present disclosure include immunomodulatory (IMiDs) compounds lenalidomide with a water solubility of less than about 1 mg/mL and melting point of 265° C. to 270° C., pomalidomide with a water solubility of less than about 1 mg/mL and melting point of about 318° C., or iberdomide with a water solubility of less than about 2 mg/mL and melting point of about 335° C. Other APIs pertinent to the present disclosure include dexamethasone with a water solubility of less than 1 mg/mL and a melting point of about 260° C. or dexamethasone acetate with a water solubility of less than 1 mg/mL and a melting point of about 263° C., and other steroids or hormones with similar physicochemical characteristics. Even further, APIs such as olanzapine with a water solubility of less than about 0.1 mg/mL and a melting point of 195° C. or risperidone with a water solubility of less than about 1 mg/mL and a melting point of about 172° C. and other CNS/antipsychotics/tricyclics with similar physicochemical characteristics, and ibrutinib with a water solubility of less than about 0.1 mg/mL and a melting point of 149° C. to 158° C. or acalabrutinib with a water solubility of less than about 0.1 mg/mL and a melting point of about 133° C., and other Bruton's tyrosine kinase (BTK) inhibitors with similar physicochemical characteristics are contemplated by the present disclosure.

Because of the low solubility of the aforementioned APIs, there is a need to develop transdermal drug delivery systems that address the availability of these drugs by modifying how the drug is solubilized and/or suspended at a molecular level in a formulation and to allow for these compounds to permeate at an appreciable rate above and beyond the permeation rates associated with certain APIs in a traditional solubilized state, so that they can be effectively delivered through the skin to achieve a continuous low-dose exposure. The transdermal delivery of these types of compounds is expected to overcome deficiencies in other routes of administration, such as, oral or IV bolus administration, where such deficiencies include but are not limited to toxicity or the peaks (above therapeutic values) and valleys (sub-therapeutic values) of pharmacokinetic profiles after administration.

Transdermal delivery of APIs is well-known for compounds that exhibit very low water solubility, such as, estradiol, testosterone, buprenorphine, fentanyl, and granisetron in a solubilized drug-in-adhesive platform. These drug-in-adhesive platforms include commercially available and marketed API products, such as Vivelle Dot® for estradiol (water solubility less than 1 mg/mL and melting point of 173° C. to 180° C.), Testoderm® for testosterone (water solubility less than 1 mg/mL and melting point of 153° C. to 155° C.), BuTrans® for buprenorphine (water solubility less than 1 mg/mL and melting point of 219° C.), generic Fentanyl Transdermal Systems for fentanyl (water solubility of about 0.2 mg/mL and melting point of 181° C. to 183° C.), and Sancuso® for granisetron (water solubility less than 0.1 mg/mL and melting point of 226° C.). These commercial transdermal delivery systems incorporate the drug in a solubilized drug-in-adhesive matrix and maintain its solubility throughout the shelf life and intended application period. It has been shown that known APIs with solubility within these matrices are maintained at or close to the saturation solubility of the matrix to obtain maximal permeation from the transdermal system (e.g., maintain a constant concentration gradient to achieve sustainable delivery). It is known that transdermal drug delivery systems are typically available in a solubilized drug-in-adhesive formulation in their simplest formulations. With APIs exhibiting challenging solubility and permeability requirements such as those referenced above, modifications to the formulations are needed to maintain the API in solution, alternative pathways to solubility must be provided for upon application, and/or specific permeability enhancers are required to try to increase the permeability of the drug molecules, all of which can complicate the path towards a commercial product with additional costs, testing, and approval requirements. In consideration of the aforementioned problems, a need exists for a transdermal drug delivery system where the permeation of low water solubility APIs is improved.

### SUMMARY OF THE PRESENT DISCLOSURE

In accordance with one embodiment of the present disclosure, a transdermal drug delivery system is disclosed. The transdermal drug delivery system includes a molecular solid suspension (MSS) of a drug in adhesive layer including an active pharmaceutical ingredient having a water solubility of less than about 10 milligrams per milliliter and a melting point greater than about 120° C., an adhesive polymer, an insoluble carrier excipient, and a surfactant. Further, a weight ratio of an amount of the active pharmaceutical ingredient suspended in the molecular solid suspension (MSS) of the drug in adhesive layer to the amount of the active pharmaceutical ingredient solubilized in the drug-in-adhesive layer is from about 1.2:1 to about 1000:1.

In another embodiment, the active pharmaceutical ingredient can have a log P value ranging from about −2 to about 8, such as between about −2 to about 1 or from about 3 to about 8, excluding log P values between about 1 and about 3, which is contradictory to the currently accepted knowledge for those skilled in the art that a log P between 1 and 3 is preferable. Not being bound by physicochemical characteristics, a log P value in the ranges described above in presence of an extreme melting point or sufficiently low water solubility may correlate to an amenable API suitable for the application of the present disclosure.

In still another embodiment, a weight ratio of the active pharmaceutical ingredient to the insoluble carrier excipient can range from about 1:0.4 to about 1:5. Further, the insoluble carrier excipient can include crosslinked polyvinylpyrrolidone, silicon dioxide, calcium silicate, or a combination thereof.

In yet another embodiment, the active pharmaceutical ingredient can be an immunomodulatory (IMiD) agent, a steroid, a hormone, or a central nervous system (CNS) agent, such as an antipsychotic or a tricyclic antidepressant.

In one more embodiment, the adhesive polymer can include an acrylate copolymer, an ethylene-vinyl acetate copolymer, a vinyl acetate-acrylic copolymer, a rubber co-polymer, a polyisobutylene polymer, a silicone polymer, or a combination thereof.

In an additional embodiment, the system can further include a humectant that can include a sugar, a sugar alcohol, a sugar ester, a polyol, phytantriol, pantothenic acid, urea, tocopherol polyethylene glycol succinate, a polyethylene glycol, hyaluronic acid, salicylic acid or derivative thereof, an alpha hydroxy acid or derivative thereof, an amino acid or derivative thereof, panthenol, or a combination thereof.

In another embodiment, the surfactant can include a first nonionic surfactant with a hydrophilic to lipophilic balance (HLB) value of less than about 12. Further, the first nonionic surfactant can include an ethoxylated alcohol. For example, the first nonionic surfactant can be Steareth-2, Oleth-2, Ceteth-3, Oleth-3, C12-13 Pareth-3, Oleth-5, C12-13 Pareth-4, Laureth-4, Ceteareth-6, Oleth-10, Steareth-10, or a combination thereof.

In addition, the nonionic surfactant can also include a second nonionic surfactant with a hydrophilic to lipophilic balance (HLB) value of greater than about 18. For example, the second nonionic surfactant can an ABA-type co-polymers of poly(ethylene oxide) (PEO=A) and poly(propylene oxide) (PPO=B). In one embodiment, the second nonionic surfactant can be a poloxamer. For instance, the second nonionic surfactant can be Poloxamer 181, Poloxamer 188, Poloxamer 338, Poloxamer 407, or a combination thereof.

In one more embodiment, the system can include an occlusive backing layer, wherein the occlusive backing layer forms an exterior facing-surface of the transdermal drug delivery system; and a release liner, wherein the release liner is positioned adjacent a skin contacting surface of the molecular solid suspension (MSS) drug in adhesive layer.

A method of forming a molecular solid suspension (MSS) of a drug in adhesive layer for a transdermal drug delivery system is also contemplated. The method includes solubilizing an active pharmaceutical ingredient in a first process solvent to form a solution, wherein the first process solvent comprises a polar aprotic solvent; adding an insoluble carrier excipient to a second process solvent to form a dispersion of particles, wherein the second process solvent is different from the first process solvent; combining the solution with the dispersion of particles to form a formulation; adding a surfactant to the formulation; and adding an adhesive polymer to the formulation, wherein a weight ratio of an amount of the active pharmaceutical ingredient suspended in the molecular solid suspension (MSS) of the drug in adhesive layer to an amount of the active pharmaceutical ingredient solubilized in the drug in adhesive layer is from about 1.2:1 to about 1000:1.

In one embodiment, the method can include coating the formulation onto one of a backing layer or a release liner.

In an additional embodiment, the method can include evaporating the process solvents.

In still another embodiment, the method can further include applying the other of the backing layer or the release liner onto an exposed surface of the formulation.

In yet another embodiment, the active pharmaceutical ingredient can have a water solubility of less than about 10 milligrams per milliliter, a melting point greater than about 120° C., and a log P value ranging from about −2 to about 8, and preferably from about −2 to about 1.0 and from about 3 to about 8, which is contradictory to the currently accepted knowledge for those skilled in the art that a log P value between about 1 and about 3 is preferable.

In one more embodiment, a weight ratio of the active pharmaceutical ingredient to the insoluble carrier excipient can range from about 1:0.4 to about 1:5. Further, the insoluble carrier excipient can include crosslinked polyvinylpyrrolidone.

In another embodiment, the active pharmaceutical ingredient can be an immunomodulatory (IMiD) agent, a steroid, a hormone, a CNS agent, such as an antipsychotic or a tricyclic antidepressant.

In still another embodiment, the adhesive polymer can include an acrylate copolymer, a polyisobutylene, a silicone, or a combination thereof.

In yet another embodiment, the surfactant can include a first nonionic surfactant having a hydrophilic to lipophilic balance (HLB) value of less than about 12. In addition, the surfactant can also include a second nonionic surfactant having a hydrophilic to lipophilic balance (HLB) value of greater than about 18.

In an additional embodiment, the method can further include adding a humectant, wherein the humectant comprises a sugar, a sugar alcohol, a sugar ester, a polyol, phytantriol, pantothenic acid, urea, tocopherol polyethylene glycol succinate, a polyethylene glycol, hyaluronic acid, salicylic acid or derivative thereof, an alpha hydroxy acid or derivative thereof, an amino acid or derivative thereof, panthenol, or a combination thereof.

A method of delivering an active pharmaceutical ingredient to a wearer via a transdermal drug delivery system is also provided. The transdermal drug delivery system includes a molecular solid suspension of a drug in adhesive layer that includes the active pharmaceutical ingredient, wherein the active pharmaceutical ingredient has a water solubility of less than about 10 milligrams per milliliter and a melting point greater than about 120° C., an adhesive polymer, an insoluble carrier excipient, and at least one nonionic surfactant, wherein a weight ratio of an amount of the active pharmaceutical ingredient suspended in the molecular solid suspension of the drug in adhesive layer to the amount of the active pharmaceutical ingredient solubilized in the drug-in-adhesive layer is from about 1.2:1 to about 1000:1. The method of delivery includes: microneedling a surface of skin of the wearer with a dermally-applied device; removing a release liner on the transdermal drug delivery system to expose a skin contacting surface of the molecular solid suspension drug in adhesive layer; and positioning the skin contacting surface of the molecular solid suspension drug in adhesive layer to the surface of skin of the wearer.

Other features and aspects of the present disclosure are set forth in greater detail below.

### BRIEF DESCRIPTION OF THE DRAWINGS

A full and enabling disclosure of the present disclosure, including the best mode thereof to one skilled in the art, is set forth more particularly in the remainder of the specification, including reference to the accompanying figure, in which:

FIG. 1 is a cross-sectional view of a transdermal drug delivery system according to one embodiment of the present disclosure, where the transdermal drug delivery system includes a molecular solid suspension (MSS) of an API seeded onto an insoluble carrier excipient;

FIG. 2 is a flow chart illustrating a method of making the transdermal drug delivery system of FIG. 1 in which the particulate of the molecular solid suspension is formed at or about Step 203 and maintained through Step 208 until solvent evaporation is complete in which the final molecular solid suspension is formed a in solvent-free polymer/adhesive layer;

FIG. 3 is a graph describing the average flux (micrograms/square centimeter/hour) of olanzapine through Strat-M® (synthetic membrane) for various transdermal drug delivery system formulations;

FIG. 4 is a graph describing the cumulative permeation (micrograms/square centimeter) of olanzapine through Strat-M® (synthetic membrane) for various transdermal drug delivery system formulations;

FIG. 5 is a graph describing the average flux (micrograms/square centimeter/hour) of ibrutinib through Strat-M® (synthetic membrane) for various transdermal drug delivery system formulations;

FIG. 6 is a graph describing the cumulative permeation of ibrutinib (micrograms/square centimeter) through Strat-M® (synthetic membrane) for various transdermal drug delivery system formulations;

FIG. 7 is another graph describing the average flux (micrograms/square centimeter/hour) of ibrutinib through Strat-M® (synthetic membrane) for various transdermal drug delivery system formulations;

FIG. 8 is another graph describing the cumulative permeation (micrograms/square centimeter) of ibrutinib through Strat-M® (synthetic membrane) for various transdermal drug delivery system formulations;

FIG. 9 is a graph describing the average flux (micrograms/square centimeter/hour) of dexamethasone through Strat-M® (synthetic membrane) for various transdermal drug delivery system formulations; and

FIG. 10 is a graph describing the cumulative permeation (micrograms/square centimeter) of dexamethasone through Strat-M® (synthetic membrane) for various transdermal drug delivery system formulations;

FIG. 11 is a graph describing the average flux (micrograms/square centimeter/hour) of lenalidomide with and without panthenol through Strat-M® (synthetic membrane) for various transdermal drug delivery system formulations;

FIG. 12 is a graph describing the cumulative permeation (micrograms/square centimeter) of lenalidomide with and without panthenol through Strat-M® (synthetic membrane) for various transdermal drug delivery system formulations;

FIG. 13 is another graph describing the average flux (micrograms/square centimeter/hour) of lenalidomide with and without panthenol through Strat-M® (synthetic membrane) for various transdermal drug delivery system formulations;

FIG. 14 is a graph describing the cumulative permeation (micrograms/square centimeter) of lenalidomide with and without panthenol through Strat-M® (synthetic membrane) for various transdermal drug delivery system formulations;

FIG. 15 is a graph describing the average flux (micrograms/square centimeter/hour) of lenalidomide with and without panthenol through Strat-M® (synthetic membrane) for various transdermal drug delivery system formulations;

FIG. 16 is a graph describing the cumulative permeation (micrograms/square centimeter) of lenalidomide with and without panthenol through Strat-M® (synthetic membrane) for various transdermal drug delivery system formulations;

FIG. 17 is a graph describing the average flux (micrograms/square centimeter/hour) of lenalidomide with and without panthenol through Strat-M® (synthetic membrane) for various transdermal drug delivery system formulations that include a nonionic surfactant having a hydrophilic to lipophilic balance (HLB) value below 12 (e.g., O3) with and without a nonionic surfactant having an HLB value greater than 12 (e.g., P407, with an HLB of 18-23);

FIG. 18 is a graph describing the average flux (micrograms/square centimeter/hour) of lenalidomide with varying concentrations of a lower HLB value (less than 12) nonionic surfactant through human cadaver skin for various transdermal drug delivery system formulations;

FIG. 19 is a graph describing the average flux (micrograms/square centimeter/hour) of lenalidomide with varying concentrations of a lower HLB value (less than 12) nonionic surfactant through Strat-M® (synthetic membrane) for various transdermal drug delivery system formulations;

FIG. 20 is a graph describing the average flux (micrograms/square centimeter/hour) of lenalidomide through human cadaver skin for various transdermal drug delivery system formulations that include nonionic surfactants having hydrophilic to lipophilic balance (HLB) values below 12 (e.g., O3 and O5) and above 12 (e.g., O10 and O20);

FIG. 21 is a graph describing the average flux (micrograms/square centimeter/hour) of lenalidomide through Strat-M® (synthetic membrane) for various transdermal drug delivery system formulations that include nonionic surfactants having hydrophilic to lipophilic balance (HLB) values below 12 (e.g., O3 and O5 both alone and in combination) with and without a poloxamer-based nonionic surfactant having a higher HLB value greater than 15 (e.g., P407, with an HLB of 18-23);

FIG. 22 is a graph describing the average flux (micrograms/square centimeter/hour) of lenalidomide for various molecular solid suspensions contemplated by the present disclosure with and without the use of a 0.5 mm and a 1.5 mm derma roller for microneedling; and

FIG. 23 is a graph describing the cumulative permeation (micrograms/square centimeter) of lenalidomide for various molecular solid suspensions contemplated by the present disclosure with and without the use of a 0.5 mm and a 1.5 mm derma roller for microneedling.

Repeat use of reference characters in the present specification and drawing is intended to represent the same or analogous features or elements of the present disclosure.

### DETAILED DESCRIPTION OF REPRESENTATIVE EMBODIMENTS

It is to be understood by one of ordinary skill in the art that the present discussion is a description of exemplary embodiments only and is not intended as limiting the broader aspects of the present disclosure.

### Definitions

As used herein, the terms “about,” “approximately,” or “generally,” when used to modify a value, indicates that the value can be raised or lowered by 5% and remain within the disclosed embodiment. Further, when a plurality of ranges are provided, any combination of a minimum value and a maximum value described in the plurality of ranges are contemplated by the present disclosure. For example, if ranges of “from about 20% to about 80%” and “from about 30% to about 70%” are described, a range of “from about 20% to about 70%” or a range of “from about 30% to about 80%” are also contemplated by the present disclosure.

As used herein, the term “colloid” generally refers to a micro-heterogeneous dispersed system in which the particle size of the dispersed phase particles is within the range 1 nanometer (nm) to 1000 nm. The phases of the colloid cannot be separated under gravity, centrifugal or other forces. The dispersed phase particles of the colloid may be separated from the dispersion medium (continuous phase) by micro-filtration. A lyophilic colloid is liquid loving and readily dispersed by a solvent and not easily precipitated, has a dispersed phase with a strong affinity for the continuous phase, and the phases interact with each other. A lyophobic colloid is liquid hating and has a dispersed phase with little or no affinity for the continuous phase, and the phases do not interact with each other. A hydrophilic colloid is water loving and has a tendency to mix with, dissolve in, or be wetted by water. A hydrophobic colloid is water hating and has a tendency to repel or fail to mix with water. A lipophilic colloid is oil loving and has a tendency to combine with or dissolve in lipids or fats. A lipophobic colloid is oil hating and includes substances that will not dissolve in or associate with lipids (fats), which are non-polar molecules, such as water.

As used herein, the term “disperse” means to cause to break up and/or to cause to become spread widely.

As used herein, the term “dispersion” means a system in which distributed particles of one material are dispersed in a continuous phase of another material. The two phases may be in the same or different states of matter.

As used herein, the term “molecular dispersion” means a true solution of a solute phase in a solvent. The dispersed phase (solute) is in the form of separate molecules homogeneously distributed throughout the dispersion medium (solvent). The molecule size is less than 1 nm.

As used herein, the term “molecular solid suspension” means the term “molecular solid suspension” shall refer specifically to a solid, such as an API, dissolved by a suitable first solvent as a true solution (molecular dispersion), however, it is incorporated into a combination of a suitable second solvent and a suitable solid excipient to serve as a carrier of the first solid. The solid excipient carrier is not soluble in the first or second solvent, however, freely disperses or suspends, dependent on particle size of substrate, in one or both solvents in combination. The solid excipient carrier may or may not be micronized. Solid excipient carriers, such as Parteck SLC (silicon dioxide) or Kollidon CL-M (crospovidone) have found suitability in preparation of these Molecular Solid Suspensions. For the present disclosure, the particle size of about no more than about 180 micrometers (μm) (Mesh #80) particle size (D90) to ensure processability of the final formulation as a transdermal delivery system. The solubilized solid in the first process solvent is in most cases an API and is added to the dispersed or suspended solid carrier, in most cases a pharmaceutically acceptable excipient, in a second process solvent (and not the first process solvent). Additionally, excipients which are amenable to production of a homogeneous dispersion are added to modify availability and permeation of API from the platform. Thus, the medium is a homogeneous blend of excipients, adhesive, polymer, or solvent such that the resulting formulation is a suspension of dispersed particles within said medium. By solvent evaporation, a viscous liquid remains, such as a pressure sensitive adhesive, which incorporates the medium and uniformly suspended particles or co-particles of API, carrier excipient, and other excipients, if needed. The API and or particle excipients may be amorphous, amorphous-like, partially amorphous, crystalline, or combinations thereof to produce a suitable formulation. The formulary of combinations of drug concentrations, significantly above solubilized saturation point, drive availability of drug to solubilize in water-based solution, and more specifically the increase in available drug is now more permeable due to enhanced concentration gradient and innate reservoir of the molecular solid suspension. As known to those skilled in the art, a molecular dispersion is a true solution and a dispersion typically results in a physical mixture of a solid material dispersed in a liquid, such as a molecular dispersion, colloid or suspension.

As used herein, the term “suspension” means a coarse dispersion which is a heterogeneous dispersed system in which the dispersed phase particles are larger than 1000 nm (1 μm). Coarse dispersions are characterized by relatively fast sedimentation of the dispersed phase caused by gravity or other forces. The dispersed phase of coarse dispersions may be easily separated from the continuous phase by filtration. The particles may be visible to the naked eye, and the mixture is only classified as a suspension when and while the particles have not settled.

As used herein, the term “partition coefficient (P)” or “distribution coefficient (D)” means the ratio of concentrations of a compound in a mixture of two immiscible solvents at equilibrium. This ratio is therefore a comparison of the solubilities of the solute in these two liquids. The partition coefficient generally refers to the concentration ratio of un-ionized species of compound, whereas the distribution coefficient refers to the concentration ratio of all species of the compound (ionized plus un-ionized). In the chemical and pharmaceutical sciences, both phases usually are solvents. Most commonly, one of the solvents is water, while the second is hydrophobic, such as 1-octanol. Hence the partition coefficient measures how hydrophilic (“water-loving”) or hydrophobic (“water-fearing”) a chemical substance is. Partition coefficients are useful in estimating the distribution of drugs within the body. Hydrophobic drugs with high octanol-water partition coefficients are mainly distributed to hydrophobic areas such as lipid bilayers of cells. Conversely, hydrophilic drugs (low octanol/water partition coefficients) are found primarily in aqueous regions such as blood serum. The partition coefficient, abbreviated P, is defined as a particular ratio of the concentrations of a solute between the two solvents (a biphase of liquid phases), specifically for un-ionized solutes, and the logarithm of the ratio is thus log P. When one of the solvents is water and the other is a non-polar solvent, then the log P value is a measure of lipophilicity or hydrophobicity. The defined precedent is for the lipophilic and hydrophilic phase types to always be in the numerator and denominator respectively; for example, in a biphasic system of n-octanol (hereafter simply “octanol”) and water. Formula I below demonstrates this relationship:

$$log {P}_{oct / wat} = {log}_{10} ( \frac{{[ solute ]}_{octanol}^{un - ionized}}{{[ solute ]}_{water}^{un - ionized}} ) \qquad ( I )$$

### DETAILED DESCRIPTION

In general, it has been found that solid suspensions and specifically those made in the form of a molecular solid suspension are surprisingly unique and allow key advantages over the solubilized platforms of similar composition to obtain maximum performance for permeation and sustainability of the delivery profile without losing concentration gradient. Typically, and known to those skilled in the art, solid drugs incorporated into transdermal systems are less available for solubilizing in skin or media, and as a result are less permeable than the formulated solubilized drug platforms. The limitation of these systems is drug loading to achieve theoretical delivery possible to obtain therapeutic blood levels of said drug, where continuing to increase drug loading either causes crystallization events or overwhelms the semi-permeable membrane (i.e., skin), ultimately reducing the overall delivery of the drug from a saturated solubilized system. The result of this disclosure overcomes the challenges of these solubilized drug platforms and incorporates or suspends elevated levels of the available drug in a molecular solid suspension that allows for a significant increase in the ability to load drug into the platform without the detrimental impact of an over saturated system as compared to a super saturated system, where the concentration of the drug is above the solubilizing limit (more than supersaturated, where supersaturated means that the drug is still solubilized) but is not so overly saturated as to be crystalline. Those molecules with intermediate to high water solubility, such as those listed in the United States Pharmacopeia (USP) as soluble to very soluble or greater than about 100 mg/mL and some molecules that are sparingly soluble or greater than about 30 mg/mL which exhibit low melting points, less than about 120° C., are less amenable to a molecular solid suspension, however, it is possible to load into the system these molecules and potentially make them more amenable to availability and thus permeability. Liquid APIs, at about ambient or room temperature, are typically incorporated within a liquid system at or above their saturation level to optimize availability and permeability which may not add significant value, although, the scientific basis is justified for improvement.

Surprisingly, it was found and it is possible through the current disclosure to exceed saturation of the API (e.g., oversaturate) in the transdermal drug delivery system and make readily available the API for permeation through skin, which is not typical of particulate drug products in transdermal systems. The type of saturation is not under saturated, at saturation level or even super saturated wherein the API is still solubilized at a level above the saturation point. Rather, the API is oversaturated but not to the level of being crystalline. In these solubilized drug systems, the soluble API, when exposed to water by hydration after application of an occlusive patch to the skin, more often than not, tends to crystallize the low water-soluble API when water is absorbed by the adhesive matrix. This crystallization event typically has a deleterious outcome on permeation and overall permeation over the entire wear period, typically up to 7 days. The API in the current disclosure, is actually present in an over-saturated level, this is done purposefully to be well above the ability of the drug in adhesive layer to solubilize to a significant extent the amount of API contained within the system and forces particulates of the API to form. How these particles form is critical to the current disclosure and maintaining these particles allows the drug to be available, soluble, and permeable after application to the skin in an occlusive system. These particles may be present in crystalline form, non-crystalline form, co-crystalline form, amorphous form, partial amorphous form, or a combination thereof. Complete amorphicity of the API is not a requirement of the formulation to be effective at permeation, however, over-saturated levels of the API are preferred to optimize delivery and performance.

More specifically, the present disclosure focuses on forming molecular solid suspensions by dissolving an API within a suitable solvent and incorporating the solubilized API into an insoluble carrier excipient and then a polymer carrier, typically a pressure sensitive adhesive, with or without additional excipients, and allowing particulates to form within the system in a uniform and consistent manner to create a suspension of suitable particles which are uniformly dispersed. The resulting molecular solid suspension of the current disclosure is a dissolved API added into a solvent dispersion of a solid, insoluble carrier excipient, such as Polyplasdone™ or Kollidon CL-M™ (crospovidone) from Ashland or BASF, respectively, which is not soluble in either process solvent nor the API, but has affinity for the API through hydrogen bonding, London dispersion forces, and or Van der Vaals forces. The API is not covalently bound to the substrate. The resulting API/solid excipient particles that are suspended in the process solvents are then added to an excipient formulation or the excipient formulation is added to the API/solid excipient particles that are suspended in the process solvents in order to maintain the API/solid excipient particles intact and keep the particles suspended during the production process into a finished dosage form such that settling does not occur during production processes. The formation of a molecular dispersion in which the API is completely solubilized by the medium does not take place as the intent of the molecular solid suspension ensures suspended particles are present in the formulation. Thus, the molecular dispersion or true solution of the API is present, initially, and then when added to a dispersed or suspended insoluble carrier excipient carrier, a molecular solid suspension is formed. The API/solid excipient particles are then uniformly dispersed within the medium. As such, a drug-in-adhesive with suitable excipients that does not interfere with the dispersive aspects of the API/solid excipient particles but contributes to the enhancement of availability and thus permeability of the API, is achieved.

Transdermal Drug Delivery System with a Molecular Solid Suspension Drug in Adhesive Layer

As mentioned above, the present disclosure is directed to a transdermal drug delivery system for the delivery of an API through the skin. In one particular embodiment, API can be an immunomodulatory agent such as lenalidomide, although it is to be understood that in alternative embodiments, any other active pharmaceutical ingredient with low water solubility less than about 10 mg/mL and a high melting point higher that about 120° C. can be utilized in the transdermal drug delivery system. Pertinent APIs evaluated in the current disclosure include IMiDs, such as lenalidomide, pomalidomide, and iberdomide, as well as steroids such as, dexamethasone and dexamethasone acetate, BTK inhibitors such as, ibrutinib, and CNS agents such as, olanzapine and risperidone. The transdermal drug delivery system includes a molecular solid suspension drug in adhesive layer that includes the API, an insoluble carrier excipient, a nonionic surfactant or combination of nonionic surfactants, a humectant, and a pressure sensitive adhesive. The transdermal drug delivery system may also include other excipients as discussed below. Further, a method of forming the molecular solid suspension drug in adhesive layer can utilize one or more polar aprotic solvents to ensure that the API is first dissolved in the solvent to form a true solution before combining the resulting solution with a solvent dispersion that includes an additional solvent distinct from the one or more polar aprotic solvents and the insoluble carrier excipient. Using this method, the insoluble carrier excipient, which is not soluble in either the additional solvent nor the API solvent, but has affinity for the API through hydrogen bonding, London dispersion forces, and or Van der Vaals forces, remains intact in particle form, and the particles remain suspended in the solvent during the production process and into a finished dosage form such that settling does not occur during the production process. Without intending to be limited by any particular theory, the present inventors have found that the specific components described above and the ratios of the components with respect to each other enhance permeation of the API through the skin.

More specifically, the present disclosure results in significantly improved performance characteristics for new and existing commercially available APIs having certain water solubility and melting temperature ranges. Permeation has shown to have improved benefits utilizing the molecular solid suspensions contemplated herein to promote an increase in rate of API delivery as well as sustainability of the API delivery profile over up to about 7 days. Stability of the API in a solubilized system is sometimes not trivial to overcome and the API may degrade to an unacceptable level over the shelf-life of the product and or the concentration gradient is lost due to patch efficiency and solubility issues after application. Thus, the molecular solid suspension formulation of the current disclosure maintains the API in a solid state which improves the ability of the API to remain stable during shelf-life storage and protects against environmental conditions such as oxidation or hydrolysis during storage as solid APIs are typically more stable than their solubilized solutions of the same API. Surprisingly, the molecular solid suspension of the current disclosure makes the API more readily available for permeation and facilitates the maintenance of a constant concentration gradient by oversaturating the drug-in-adhesive matrix in a uniform and consistent manner. For instance, a weight ratio of an amount of active pharmaceutical ingredient suspended in the molecular solid suspension of the drug in adhesive layer (such as on the insoluble carrier excipient and/or other excipients) to an amount of active pharmaceutical ingredient solubilized in the drug in adhesive layer can range from about 1.2:1 to about 1000:1, such as from about 1.5:1 to about 500:1, such as from about 2:1 to about 100:1, and any ratios therebetween. It should be understood that such a ratio results in an oversaturation of the API in the molecular solid suspension of the drug in adhesive layer rather than a supersaturation of the API in the molecular solid suspension of the drug in adhesive layer, where such oversaturation keeps the concentration of the API above the solubilizing limit of the API, yet the API is not in a crystalline form.

Relevant to the present disclosure, transdermal delivery of an API with a negative Log P is outside the scope of Lipinski's Rule of 5, where this famous “rule of 5” has been highly influential in API development, but only about 50% of orally administered new chemical entities obey it. The rule is important to keep in mind during API discovery when a pharmacologically active lead structure is optimized stepwise to increase the activity and selectivity of the compound as well as to ensure API-like physicochemical properties are maintained as described by Lipinski's rule. Candidate APIs that conform to the “rule of 5” tend to have lower attrition rates during clinical trials and hence have an increased chance of reaching the market. The rule of 5 is not a set of 5 rules, however each rule is divisible by 5. As a general rule of thumb, a negative Log P implies the molecule should exhibit higher water solubility (hydrophilicity) as compared to being soluble in the organic phase (lipophilicity). Some molecules close to Log P from about 0 to about −1.5 exhibit unexpected solubilities and experimental solubility may not be properly determined by Log P estimation alone.

TABLE 4
Lipinski's Rule of 5

| Rules | Criteria |
|---|---|
| Hydrogen Bond Donors (—OH or —NH) | NMT 5 |
| Hydrogen Bond Acceptors (N or O) | NMT 10 |
| Molecular Weight (Daltons) | NMT 500 |
| Log P (#) | NMT 5 |

Modifications to the rules throughout the years include the Ghose filter, Veber's rules, and other notable changes, such as, Log P from −0.4 to +5.6; molar refractivity from 40 to 130; molecular weight from 180 to 480; and # of atoms from 20 to 70 which includes donor and acceptors in totality rather than independently accounting for them as a rule #1 or #2. Rotatable bonds less than 10 and polar surface area no greater than 140 A² have been found to have good oral bioavailability. These rules have been found to correlate to transdermal delivery of APIs as well to show viability of delivery.

TABLE 5
Ghose Filter and Veber's Rule Sets

| Rules | Criteria |
|---|---|
| Molecular Weight (Daltons) | Range 180 to 480 |
| Log P (#) | Range −0.4 to +5.6 |
| Molar Refractivity | 40 to 130 |
| # of atoms | 20 to 70 atoms |
| Rotatable Bonds | NMT 10 |
| Polar Surface Area | NMT 140 A² |

Of particular interest, the present inventors have found that the APIs contemplated by the present disclosure may be outside the scope of Lipinski's rule of 5, Ghose filter, or Veber's rule on 1 or more key attributes, which would indicate that successful transdermal delivery is not viable; yet, the methods and formulations of the present disclosure show that transdermal delivery with increased permeation through the skin can be achieved.

Interestingly enough, melting point and water solubility are left out of these general rules of thumb as it comes to develop of APIs for drug delivery. The present inventors recognized APIs which exhibit characteristics for low water solubility (less than about 10 mg/mL) and have relatively high melting points (greater than about 120° C.) are preferable to the current disclosure as molecular solid suspensions of solid APIs are obtained. However, depending on other properties, API molecules that exhibit higher water solubility than 10 mg/mL may be amenable to formation of these molecular solid suspensions, such that melting points are greater than about 120° C.

Transdermal delivery systems (TDS) described herein include transdermal formulations which may be in form of a liquid or semi-solid form of a desired degree of viscosity, for example, a suspension, nano suspension, micro suspension, dispersion, emulsion, micro emulsion, nano emulsion, gel, ointment, cream, paste, lotion, mousse, adhesive, patch, plaster, or balm.

The transdermal molecular solid suspension formulation may be the entire system or may form a part of a TDS system that comprises the transdermal molecular solid suspension formulation as a layer, for instance. Exemplary TDS include, without limitation, topical or transdermal formulations, systems, patches, or matrices in a bi-layered, multilayered or monolithic system, with or without adhesive, with or without overlay, as a drug-in-adhesive, reservoir, microreservoir, hydrogel, mucoadhesive, adhesive, tape, microneedle, microblade, micro protrusion, dissolvable microneedle, absorbable microneedle, as a system, patch, plaster, or combinations thereof for topical or transdermal use.

In further embodiments, the formulations provided herein provide for stable formulations of the API in the formulations. For example, the formulations are shelf stable and maintain at least 90% of their activity over a predetermined period, when stored under standard ambient conditions. In still further embodiments, the formulations are shelf stable for at least 3 months, 6 months, 9 months, one (1) year, two (2) years, or longer.

In another embodiment and depending on the API being delivered, the average flux rate of the API included in the transdermal drug delivery systems of the present disclosure can be at least 1 μg/cm²/hr continuously throughout a period of 1, 2, 3, 4, 5, 6, 7, or more days. In some embodiments, the average flux rate of the API included in the transdermal drug delivery systems of the present disclosure can be at least 2 μg/cm²/hr continuously throughout a period of 1, 2, 3, 4, 5, 6, 7, or more days, or the average flux rate of the API included in the transdermal drug delivery systems of the present disclosure can be at least 3 μg/cm²/hr continuously throughout a period of 1, 2, 3, 4, 5, 6, 7, or more days. In still other embodiments, the average flux rate of the API included in the transdermal drug delivery systems of the present disclosure can be at least 4 μg/cm²/hr continuously throughout a period of 1, 2, 3, 4, 5, 6, 7, or more days.

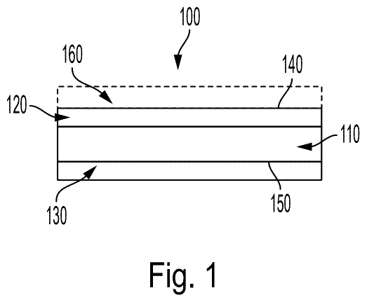

Referring to FIG. 1 and according to one particular embodiment, the transdermal drug delivery system 100 includes a molecular solid suspension of a drug in adhesive layer 110 disposed between a backing layer 120 and a release liner 130. The backing layer 120 has an exterior surface 140 that is exposed to the ambient environment when the transdermal drug delivery system 100 is in use. Meanwhile, the release liner 130 is positioned on a skin-contacting surface 150 of the molecular solid dispersion drug in adhesive matrix layer 110, where the release liner 130 is removable so that the molecular solid suspension drug in adhesive layer 110 can be positioned directly on the skin during use of the transdermal drug delivery system 100. As a result of the specific combination of components used to form the molecular solid suspension drug in adhesive layer, such as the API, particular process solvents, insoluble carrier excipient, and permeation enhancers, as well as the specific weight percentages and ratios of such components utilized, the present inventors have found that the transdermal drug delivery system 100 can include a super-saturated yet stable molecular solid suspension drug in adhesive matrix layer that forms a skin-contacting surface, which facilitates the delivery of the API in a controlled manner at elevated levels. For instance, the skin permeation of the API can be based on the ratio of the API to the insoluble solid carrier excipient (e.g., cross-linked polyvinylpyrrolidone), and/or the ratio of the API to the skin permeation enhancer. As shown in FIG. 1, the molecular solid suspension drug in adhesive layer 110 can be in the form of a single layer so that the active pharmaceutical ingredient is uniformly dispersed throughout the adhesive component of the system 100 via the insoluble carrier excipient, which maintains the API in a solid state.

The various components of the transdermal drug delivery system 100 are discussed in detail below.

### 1. Molecular Solid Suspension Drug in Adhesive Layer

a. Active Pharmaceutical Ingredient

The API component of the molecular solid suspension drug in adhesive layer of the transdermal drug delivery system of the present disclosure can be any drug or active pharmaceutical ingredient (API) that has a low water solubility and high melting point, as APIs having such properties have been found to exhibit increased permeation and stability utilizing the transdermal delivery systems of the present disclosure. For instance, the API can have a water solubility of less than about 10 mg/mL, such as less than about 5 mg/mL, such as less than about 1.5 mg/mL, such as less than about 1.25 mg/mL, such as less than about 1 mg/mL. Further, the API can have a melting point of greater than 120° C., such as a melting point ranging from about 140° C. to about 285° C., such as from about 145° C. to about 280° C., such as from about 150° C. to about 275° C., or any ranges therebetween. Moreover, the API can have a log P value ranging from about −2 to about 5, such as from about −1.75 to about 4.5, such as from about −1.5 to about 4, such as from about −0.5 to about 3, or any ranges therebetween.

In one embodiment, the API can be an immunomodulatory agent. For instance, the immunomodulatory agent can include all pharmaceutically acceptable forms of an immunomodulatory imide compound, such as thalidomide, including analogs of thalidomide including lenalidomide, pomalidomide, and iberdomide including, for example, free base, salts, polymorphs, solvates, solutions, isomers, amorphous, crystalline, co crystalline, solid solution, prodrugs, analogs, derivatives, and metabolites and combinations thereof. The compound may be in the form of a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methane sulphonate, ethanesulphonate, benzenesulphonate, p-toluenesulphonate and pamoate salts.

In another embodiment, the API can be a hormone such as a steroid. For example, the API can be a corticosteroid such as dexamethasone. Corticosteroids are a class of steroid hormones that are produced in the adrenal cortex of vertebrates, as well as the synthetic analogues of these hormones. Two main classes of corticosteroids, glucocorticoids, and mineralocorticoids, are involved in a wide range of physiological processes, including stress response, immune response, and regulation of inflammation, carbohydrate metabolism, protein catabolism, blood electrolyte levels, and behavior. Synthetic pharmaceutical drugs with corticosteroid-like effects are used in a variety of conditions, ranging from hematological neoplasms to brain tumors or skin diseases. Dexamethasone and its derivatives are almost pure glucocorticoids, while prednisone and its derivatives have some mineralocorticoid action in addition to the glucocorticoid effect. Fludrocortisone is a synthetic mineralocorticoid. Hydrocortisone is typically used for replacement therapy, e.g., for adrenal insufficiency and congenital adrenal hyperplasia. Other corticoids include budesonide, and deflazacort. In another embodiment, the API can be an androgenic steroid such as testosterone, methyltestosterone, oxymetholone, or fluoxymesterone. In still another embodiment, the API can be an estrogen such as a conjugated estrogen, an esterified estrogen, estropipate, 17-β estradiol, 17-β estradiol valerate, equilin, mestranol, estrone, estriol, ethinyl estradiol, or diethylstilbestrol. In yet another embodiment, the API can be a progestin or progestogen such as progesterone, 19-norprogesterone, norethinedrone and its derivatives, melengestrol, chlormadinone, ethisterone, medryoxyprogesterone and its derivatives, hydroxyprogesterone and its derivatives, ethynodiol diacetate, norethynodrel, 17-α hydrosxyprogesterone, dydrogesterone, dimethisterone, ethinylestrenol, norgestrel, norelgestromin, norgestimate, drospirenone, etonogestrel, levonorgestrol, desogestrel, demegestone, promegestone, or megestrol acetate. 5-alpha-reductase inhibitors include dutasteride and finasteride.

Anti-inflammatory agents, such as, hydrocortisone, cortisone, dexamethasone, triamcinolone, dexamethasone acetate and other derivatives, fluocinolone, triamcinolone, medrysone, prednisolone, flurandrenolide, prednisone, halcinonide, methylprednisolone, fludrocortisone, corticosterone, paramethasone, betamethasone and derivatives.

In yet another embodiment, the API can be an antipsychotic or tricyclic antidepressant. For instance, the API can be olanzapine (2-methyl-10-(4-methyl-1-piperazinyl)-4H-thieno-[2,3-b][1,5]benzo-diazepine), which is an antipsychotic medication used to treat schizophrenia and bipolar disorder. It is usually classed with the atypical antipsychotics, a newer generation of antipsychotics. It has been approved by the FDA in tablet form under the brand name Zyprexa® for treatment of schizophrenia and bipolar mania. Olanzapine has also been investigated for use as an antiemetic at oral doses of 10 mg and 5 mg a day, generally in combination with one or more further agents, e.g., to treat nausea and vomiting after administration of the chemotherapeutic cisplatin. Other antipsychotic APIs contemplated by the present disclosure include thiopropazate, chlorpromazine, triflupromazein, mesoridazine, piperacetazine, thioridazine, acetophenazine, fluphenazine, perphenazine, trifluoperazine, chlorprathixene, thiothixene, haloperidol, bromperidol, loxapine, molindone, aripiprazole, lurasidone, quetiapine, cariprazine, brexpiprazole, olanzapine, ziprasidone, asenapine, risperidone, paliperidone, lumateperone, iloperidone, pimavanserin, and clozapine.

In still another embodiment, the API can be a Bruton's tyrosine kinase (BTK) inhibitor. BTK inhibitors are a type of drug that work to treat cancers caused by defective B cells, such as chronic lymphocytic leukemia, B-cell lymphomas, and Waldenström macroglobulinemia. For example, the API can be ibrutinib, acalabrutinib, pirtobrutinib, zanubrutinib, evobrutinib, tirabrutinib, and orelabrutinib

Regardless of the particular API utilized, the amount of the API contained in the molecular solid suspension drug in adhesive layer can range from about 1 wt. % to about 50 wt. %, such as from about 3 wt. to about 30 wt. %, such as from about 4 wt. % to about 25 wt. %, such as from about 4.25 wt. % to about 20 wt. %, such as from about 4.5 wt. % to about 15 wt. %, such as from about 5.0 wt. % to about 10 wt. %, or any ranges therebetween, based on the dry weight of the molecular solid suspension drug in adhesive layer.

b. First Process Solvent for API

A first process solvent can be used to dissolve or solubilize the API into a true solution with the solvent. This solution, which can be referred to as the solubilized API solution, can then combined with an excipient formulation which will be discussed in more detail below. The first process solvent used to dissolve or solubilize the API can be a volatile solvent. In one embodiment, the first process solvent can include one or more polar aprotic solvents to maximize solubility and thus concentration of drug in first solvent system.

A polar aprotic solvent is a solvent that lacks an acidic proton and is polar. Such solvents lack hydroxyl and amine groups. These solvents do not serve as proton donors in hydrogen bonding, although they can be proton acceptors. Specific examples contemplated by the present disclosure can include Pharmasolve® a brand name of n-methyl-2-pyrrolidone (NMP), 2-pyrollidone (2-pyrol®), dioxane, propylene carbonate, dimethyl sulfoxide (DMSO), dimethyl isosorbide, dimethylacetamide, ethyl acetate, or a combination thereof, although it is to be understood that other polar aprotic solvents are also contemplated by the present disclosure, including, but not limited to, acetone, acetonitrile, dichloromethane, dimethylformamide, DMPU, and tetrahydrofuran.

Regardless of the particular polar aprotic solvent or combination of polar aprotic solvents utilized, the total amount of polar aprotic solvent contained in the molecular solid suspension drug in adhesive layer can be detectable in the transdermal drug delivery system in an amount less than ICH Q3C Impurities: Guideline for Residual Solvents. For NMP, this equates to levels of less than about 530 parts per million, or less than about 0.053 wt. %, such as less than about 390 parts per millions, or less than about 0.039 wt. %, based on the dry weight of the solid dispersion drug in adhesive layer where the NMP is to be considered a process solvent. However, it is to be understood that such solvents are introduced in larger wt. % levels to form the solubilized API/first process solvent solution and prior to any evaporation or drying. Moreover, it should be noted that the stable solid API uniform molecular dispersion of the present disclosure is formed by solubilizing the API in a volatile solvent at a relatively high concentration as compared to its incorporation into the system. The solubilized API is added to the adhesive blend comprising solvent-based adhesive, process solvents, and or excipients in order to produce a uniform blend where an API particle is formed in situ prior to evaporating the first process solvent and other process solvents in a continuous coating process.

c. Insoluble Carrier Excipient

The molecular solid suspension drug in adhesive layer of the transdermal drug delivery system of the present disclosure can also include an insoluble carrier excipient that has a particle size of less than about 180 micrometers that acts as a substrate for particle formation when combined with the solubilized API and first process solvent described above. In one embodiment, the insoluble carrier excipient can be a micronized crosslinked polyvinylpyrrolidone (PVP), such as a crosslinked homopolymer of N-vinyl-2-pyrrolidone, which can allow molecular adsorption of the API onto a solid porous substrate in the adhesive layer. In one particular embodiment, the crosslinked PVP is in the form of a water-insoluble powder. Such cross-linked PVPs are commercially available under the name Kollidon®, available from BASF. A specific example of a cross-linked PVP that is contemplated for use in the present disclosure is Kollidon® CL-M. Other cross-linked PVPs that can be used include Kollidon® CL-SF and CL-F, as well as Ashland Polyplasdone®. Other insoluble carrier excipients that are contemplated include cellulosic derivatives, such as ethyl cellulose, croscarmellose, carboxymethylcellulose, or starches, as well as cross-linked acrylic polymers, such as Carbopol, minerals or clays, such as silica, Polargel®, bentonite, kaolin, or silicates. In some embodiments, the insoluble carrier excipient can include silicon dioxide or calcium silicate alone or in combination with any of the aforementioned insoluble carrier excipients.

Further, the insoluble carrier excipient can be micronized, although this is not required, and can have an average particle size ranging from about 1 micrometer to about 40 micrometers, such as from about 2 micrometers to about 30 micrometers, such as from about 3 micrometers to about 10 micrometers. In addition, in one particular embodiment, greater than 90% of the particles utilized can have a particle size less than about 15 micrometers. The particle size of the cross-linked PVP contemplated for use in the drug-in-adhesive matrix layer of the present disclosure is thus smaller than typical cross-linked PVPs, which can have particle sizes up to 150 micrometers. Without intending to be limited by any particular theory, the present inventors have found that utilizing a cross-linked PVP where greater than 90% of the particles have a particle size less than about 15 micrometers can result in the formation of a stable polymer blend that is used to form the molecular solid suspension drug in adhesive layer, where the API is maintained in a uniform suspension with minimal sedimentation. This, in turn, enables the formation of a homogeneous solid dispersion of the API in the drug in adhesive layer so that the transdermal drug delivery system can deliver the API in a controlled manner through the skin, where it is understood that the insoluble carrier excipient is micronized and disperses within the solvent system such that it does not significantly dissolve or swell in the presence of the solvents, API, or other excipients, thus allowing for adsorption of the API onto the particles.

Moreover, the insoluble carrier excipient contemplated for use in the present disclosure can have a bulk density ranging from about 0.10 g/mL to about 0.40 g/mL, such as from about 0.125 g/mL to about 0.35 g/mL, such as from about 0.15 g/mL to about 0.25 g/mL. Further, the insoluble carrier excipient can have a surface area ranging from about 0.5 m²/g to about 20 m²/g, such as from about 1 m²/g to about 15 m²/g, such as from about 1.5 m²/g to about 10 m²/g. The increased surface area of the particles of the insoluble carrier excipient contemplated for use in the present disclosure can facilitate the dispersion of the API in a uniform, homogeneous manner throughout the molecular solid suspension drug in adhesive layer, which enables the API to be delivered at a constant rate.

The amount of the insoluble carrier excipient contained in the molecular solid suspension drug in adhesive layer can range from about 4 wt. % to about 25 wt. %, such as from about 4.25 wt. % to about 20 wt. %, such as from about 4.5 wt. % to about 15 wt. %, such as from about 5.0 wt. % to about 10 wt. %, or any ranges there between based on the dry weight of the molecular solid suspension drug in adhesive layer. Further, the present inventors have found that the ratio of the API to the insoluble carrier excipient impacts the formation of the molecular solid suspension, where a ratio of the API to the insoluble carrier excipient that is from about 1:0.4 to about 1:5, such as from about 1:0.5 to about 1:2, such as about 1:0.6 to about 1:1.5, facilitates formation of a stable, solid API molecular dispersion when the API is introduced in soluble form to the other components of the drug in adhesive layer.

d. Second Process Solvent

A second process solvent can be used to form a dispersion with the insoluble carrier excipient discussed above additional components may be added to the second process solvent, where such additional components are discussed below. The resulting formulation, which can be referred to as the excipient formulation can then combined with the solubilized API solution. Any suitable solvent can be used so long as the second process solvent is different than the first process solvent and so long as the insoluble carrier excipient is not capable of being dissolved therein since the insoluble carrier excipient serves as a particle substrate for in situ formation of the API/insoluble carrier excipient particles that are ultimately dispersed homogeneously within the resulting transdermal drug delivery system.

In one embodiment, the second process solvent is typically different from the first process solvent, where the drug is not necessarily soluble to a significant extent in the second solvent. It may comprise similar solvents such as a polar aprotic solvent. Specific examples contemplated by the present disclosure can include Pharmasolve®, a brand name of n-methyl-2-pyrrolidone (NMP) as well as any other suitable NMP, 2-pyrollidone (2-pyrol®), dioxane, propylene carbonate, dimethyl sulfoxide (DMSO), dimethyl isosorbide, dimethylacetamide, ethyl acetate, or a combination thereof, although it is to be understood that other polar aprotic solvents are also contemplated by the present disclosure, including, but not limited to, acetone, acetonitrile, dichloromethane, dimethylformamide, DMPU, and tetrahydrofuran.

Still other solvents contemplated include heptane, hexane, propyl acetate, butyl acetate, isopropyl alcohol, ethanol, cyclohexane, volatile silicone fluids, and toluene.

Regardless of the particular solvent or combination solvents utilized, the total amount of polar aprotic solvent contained in the resulting molecular solid suspension drug in adhesive layer can be detectable in the transdermal drug delivery system in an amount less than ICH Q3C Impurities: Guideline for Residual Solvents. For NMP, this equates to levels of less than about 530 parts per million, or less than about 0.053 wt. %, such as less than about 390 parts per millions, or less than about 0.039 wt. %, based on the dry weight of the molecular solid suspension drug in adhesive layer where the NMP is to be considered a process solvent. For Class 3 solvents, to be considered a process solvent, the solvent is typically present in an amount of less than 5000 parts per million or less than 0.5 wt. %, such as less than about 3900 parts per million, or less than about 0.39% wt. %, based on the dry weight of the molecular solid suspension drug in adhesive layer. However, it is to be understood that such solvents are introduced in larger wt. % levels to form the excipient formulation and prior to any evaporation or drying.

e. Adhesive Polymer

The molecular solid suspension drug in adhesive layer of the transdermal drug delivery system of the present disclosure also includes one or more suitable pressure sensitive adhesives (PSA). Adhesive polymers may be made from various materials which include plastics, polymers, pressure sensitive adhesives, self-adhering systems, or may require additional excipients to obtain pressure sensitive properties. Basic adhesive systems are selected from polyacrylics, silicones, polyisobutylenes, rubbers, and combinations thereof either by physical blending or copolymerization is disclosed. These materials may be obtained from solvent-borne, water-borne, physical mixtures, extruded, co-extruded, hot melt, or otherwise formed as polymerized or unpolymerized materials.

In one embodiment, the PSA can be an acrylic polymer. Useful acrylic polymers include various homopolymers, copolymers, terpolymers and the like of acrylic acids and derivatives thereof as a cross-linked, cross-linkable, uncross-linked, uncross-linkable, grafted, block, cured and non-curing pressure sensitive adhesives (PSAs). These acrylic polymers include copolymers of alkyl acrylates or methacrylates. Polyacrylates include acrylic acid, methacrylic acid, and derivatives thereof without limitation, methyl acylate, methyl methacrylate, ethyl acrylate, ethyl methacrylate, n-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decylmethacrylate, dodecyl acrylate, dodecyl methacrylate, tridecyl acrylate, tridecyl methacrylate, vinyl acetate, 2-hydroxyethyl acrylate, glycidyl methacrylate, or octylacrylamide. The acrylic polymer may be functional species with levels of hydroxyl or carboxyl moieties or combinations thereof, non-functional species without functional moieties, non-reactive species with moieties which are less reactive than hydroxyl or carboxyl moieties, such as methyl or ethyl or propyl or butyl capped acrylamides. Exemplary acrylic PSAs include, without limitation, one or more of: Duro-Tak® 87-900A, Duro-Tak 87-9301, Duro-Tak® 87-4098, Duro-Tak® 387-2510/87-2510, Duro-Tak® 387-2287/87-2287, Duro-Tak® 87-4287, Duro-Tak® 387-2516/87-2516, Duro-Tak® 87-2074, Duro-Tak® 87-235A, Duro-Tak 387-2353/87-2353, Gelva® GMS 9073, Duro-Tak® 87-2852, Duro-Tak® 387-2051/87-2051, Duro-Tak® 387-2052/87-2052, Duro-Tak® 387-2054/87-2054, Duro-Tak® 87-2194, or Duro-Tak® 87-2196. It should also be understood that the disclosure herewith incorporates known and unknown naming conventions comprising the monomers disclosed.

In one particular embodiment, the present inventors have found that the use of a PSA that includes an acrylate copolymer without having —COOH or —OH functional groups or moieties contribute to the improved permeation of the API contained in the drug in adhesive layer. Further, it has also been found that an acrylate copolymer having a solids content ranging from about 30% to about 55%, such as from about 35% to about 50%, such as from about 36% to about 45% also contributes to the improved solubility and permeation of the immunomodulatory agent. Additionally, an acrylate copolymer having a viscosity of less than about 6500 centipoise, such as from about 2000 centipoise to about 5000 centipoise, such as from about 2500 centipoise to about 4500 centipoise may also contribute to the improved solubility and permeation of the API, where the viscosity impacts the loading capacity of the components in polymer blend used to form the drug in adhesive matrix layer. Further, an acrylate copolymer that includes vinyl acetate may also be beneficial.

Particular examples include Duro-Tak® 87-9301 (non-reactive amine, 36.5% solids), Duro-Tak® 387-2516/87-2516 (vinyl acetate; —OH functional groups; 41.5% solids; viscosity of 4350 centipoise), Duro-Tak® 387-2052/87-2052 (vinyl acetate; —COOH functional groups, 47.5% solids; viscosity of 2750 centipoise), or Duro-Tak® 87-4098 (vinyl acetate; 38.5% solids content; viscosity of 6500 centipoise).

In still another embodiment, the PSA can include silicone. Suitable silicone adhesives include pressure sensitive adhesives made from silicone polymer and resin. The polymer to resin ratio can be varied to achieve different levels of tack. Specific examples of useful silicone adhesive which are commercially available include the standard DuPont® Liveo® BIO-PSA series (7-4400, 7-4500, and 7-4600 series) and the amine compatible (end capped) DuPont® Liveo® BIO-PSA series (7-4100, 7-4200, and 7-4300 series) manufactured by DuPont. Preferred adhesives include LIVEO® versions of the well-known BIO-PSA 7-4101, 7-4102, 7-4201, 7-4202, 7-4301, 7-4302, 7-4401, 7-4402, 7-4501, 7-4502, 7-4601, and 7-4602. Soft elastomeric silicone adhesives include Dupont® Liveo® Soft Skin Adhesives such as MG7-9700 Kit (A&B), MG7-9800 Kit (A&B), MG7-9850 Kit (A&B), and MG7-9900 Kit (A&B).

In still another embodiment, the PSA can include polyisobutylene. Suitable polyisobutylene adhesives are those which are pressure sensitive and have suitable tack. The polyisobutylene can comprise a mixture of high and medium molecular weight polyisobutylenes, polybutenes, and mineral oils. Specifically, high molecular weight polyisobutylenes are those with a molecular weight of at least about 425,000. Medium molecular weight polyisobutylenes are those with a molecular weight of at least 40,000 but less than about 425,000. Low molecular weight polyisobutylenes are those with a molecular weight of at least 100 but less than about 40,000. Specific examples of useful polyisobutylene adhesives which are commercially available include Oppanol® High Molecular Weight N grades 50, 50SF, 80, 100 and 150, and Oppanol® Medium Molecular Weight B grades 10N, 10SFN, 11 SFN, 12SFN, 12N, 13SFN, 14SFN, 15SFN, and 15N manufactured by BASF. Specific examples of polybutenes are commercially available from Soltex as polybutenes of various molecular weights and by Ineos as Indopol and Panalane with various molecular weights. A specific example of a useful polyisobutylene formulated adhesive which is commercially available includes Henkel Duro-Tak® 87-6908.

Other pressure sensitive adhesives obtained from rubber block copolymers, such as Styrene-Isoprene-Styrene (SIS) or Styrene-Butadiene-Styrene (SBS) based adhesives are also contemplated by the present disclosure.

Regardless of the particular PSA utilized, the pressure sensitive adhesive can be present in an amount ranging from about 1 wt. % to about 80 wt. %, such as from about 20 wt. % to about 75 wt. %, such as from about 40 wt. % to about 70 wt. %, or any ranges therebetween, based on the dry weight of the molecular solid suspension drug in adhesive layer.

In addition, it should be understood that other polymers can be utilized as adhesive polymers in combination with a plasticizer. For instance, other polymers that can be utilized that can include an ethylene-vinyl acetate copolymer, such as Celanese® EVAs, a polyvinylpyrrolidone, such as BASF's Kollidons™ or Ashland's Plasdones®, a cellulose, such as Ashland's Aqualon™, Benece™ or Klucel™ or a combination thereof.

f. Skin Permeation and Solubility Enhancers

The molecular solid suspension drug in adhesive layer of the transdermal drug delivery system of the present disclosure can also include one or more suitable surfactants (e.g., non-ionic surfactants), plasticizers, humectants, or a combination thereof that can serve as a skin permeation enhancer to improve the permeation of the API through the skin during use of the transdermal drug delivery system.

In some embodiments, the skin permeation enhancer can include one or more of the following: sulfoxides, and similar chemicals such as but not limited to dimethyl sulfoxide, dimethylacetamide, dimethylformamide, dimethyl sulfoxide, dimethyl isosorbide; azone, pyrrolidone such as but not limited to N-methyl-2-pyrrolidone, 2-pyrrolidone, N-(2-hydroxyethyl)-2-pyrrolidone (HEP), n-octyl pyrrolidone (NOP), N-ethyl-pyrrolidone (NEP); esters, fatty acid esters such as but not limited to propylene glycol monolaurate, butyl ethanoate, ethyl ethanoate, isopropyl myristate, isopropyl palmitate, ethyl oleate, oleyl oleate, methyl ethanoate, decyl oleate, propylene glycol monocaprate, propylene glycol monolaurate, diethylene glycol monoethyl ether, glycerol monooleate, glycerol monolaurate, lauryl laurate, lauryl lactate, and others; fatty acids (C8 to C26 fatty acids), such as but not limited to capric acid, caprylic acid, lauric acid, oleic acid, myristic acid, linoleic acid, stearic acid, palmitic acid, stearic acid, arachidic acid, behenic acid, lignoceric acid, cerotic acid, myristoleic acid, palmitoleic acid, sapienic acid, elaidic acid, vaccenic acid, lenoelaidic acid, alpha-linolenic acid, arachidonic acid, eicosapentaenoic acid, erucic acid, docosahexaenoic acid, and others; fatty alcohols (C4 to C24 fatty alcohols), such as but not limited to tert-butyl alcohol, tert-amyl alcohol, 3-methyl-3-pentanol, heptanol, octanol, perlargonic alcohol, decanol, undecyl alcohol, tridecyl alcohol, pentadecyl alcohol, cetyl alcohol, palmitoleyl alcohol, heptadecyl alcohol, stearyl alcohol, oleyl alcohol, Lauryl alcohol, myristyl alcohol, nonadecyl alcohol, arachidyl alcohol, heneicosyl alcohol, behenyl alcohol, erucyl alcohol, lignoceryl alcohol, ceryl alcohol, hepacosanol, montanyl alcohol, nonacosanol, myricyl alcohol, dotriacontanol, geddyl alcohol and others.; and glycols such as but not limited to, nathanol, dodecanol, propylene glycol, dipropylene glycol, tripropylene glycol, polyethylene glycol (MW 200 to 20000), glycerol and others ethers alcohol such as but not limited to diethylene glycol monoethyl ether; urea, triglycerides such as but not limited to medium chain triglycerides (MCT), triacetin, triolein; polyoxyethylene fatty alcohol ethers, triethyl citrate, polyoxyethylene fatty acid esters, esters of fatty alcohols, essential oils, hydramol, surfactant type enhancers such as but not limited to a nonionic surfactant, such as a nonionic surfactant of a fatty alcohol, one of its derivatives, or a combination thereof. For instance, polyoxyethylene or alcohol ethoxylate surfactants based on lauryl alcohol, oleyl alcohol, or cetyl alcohol such as Brij L, LT, C, CS, O or S, such as but not limited to S2, LT3, O2, O3, O5, LT4, L4, CS6, O10, S10, CS12, L9, S20, O20, S721, CS20, CS25, LT23, or L23, where the lauryl alcohol (L) series has a C12 alkyl chain, the synthetic lauryl alcohol (LT) series has a C12-13 alkyl chain (e.g., C12-13 Pareth-3 or C12-C12 Pareth-4), the cetyl alcohol (C) series has a C16 alkyl chain, the cetearyl alcohol (CS) series has a C16-18 alkyl chain, the stearyl alcohol (S) series has a C18 alkyl chain, and the oleyl alcohol (O) series has a C18:1 alkyl chain. Meanwhile, the number after each series of letters or letter refers to the number of moles of EO present, referring to the level of ethoxylation. Specifically, non-ionic surfactants having HLB values of less than about 12, such as HLB values less than about 10, such as HLB values between about 5 and about 10, were found to be highly advantageous, including, but not limited to O3, O5, L4, S2, LT3, LT4, C2, and/or CS6. Meanwhile, non-ionic surfactants having HLB values above 12, such as O10 and O20 with HLB values of 12.4 and 15.5, respectively, were found to not be as advantageous in the particular formulations of the present disclosure in terms of improvement of the permeation of the API. Additionally, surfactants such as sodium lauryl sulfate, tween, polysorbate; terpene, terpenoids and all penetration or permeation enhancers referred in the book “Percutaneous Penetration Enhancers” (Eric W. Smith, Howard I. Maibach, 2005. Nov, CRC press) are contemplated by the present disclosure.

For example, nonionic surfactants that can be utilized include oleths that include one or more polyethylene glycol ethers of oleyl alcohol and laureths that include one or more polyethylene glycol ethers lauryl alcohol. For instance, the skin permeation enhancer can be polyethylene glycol dodecyl ether (Brij L4 or laureth-4). Without intending to be limited by any particular theory, is believed that the nonionic surfactant contributes to an increase in flux and the ability of the system to overcome a barrier of drop in flux 24-hours post application to the skin.

Other non-ionic surfactants that are contemplated are ABA-type co-polymers of poly(ethylene oxide) (PEO=A) and poly(propylene oxide) (PPO=B), which can be referred to as poloxamers (e.g., P181 (HLB of 29), P188 (HLB>24), P338 (HLB>24), P407 (HLB of 18-23), or a combination thereof, commercially available as Kolliphor®, Pluronic®, or Lutrol®), or any other suitable surfactant having an HLB value of greater than about 18, such as from about 18 to about 32, such as from about 18 to about 30, which can act as a solubility enhancer when used in combination with a nonionic surfactant having an HLB of less than about 12.

Regardless of the particular skin permeation enhancer utilized, the skin permeation enhancer can be contained in a polymer blend used to form the drug in adhesive layer and can be present in an amount ranging from about 5 wt. % to about 40 wt. %, such as from about 10 wt. % to about 37.5 wt. %, such as from about 15 wt. % to about 35 wt. %, based on the dry weight of the molecular solid suspension drug in adhesive layer of the transdermal drug delivery system. In one particular embodiment, the skin permeation enhancer can include from about 2.5 wt. % to about 30 wt. %, such as from about 5 wt. % to about 22.5 wt. %, such as from about 7.5 wt. % to about 25 wt. % of a first non-ionic surfactant (e.g., one or more polyethylene glycol ethers of oleyl alcohol such as oleth-3 in combination with oleth-5, or laureth-4) and from about 0.5 wt. % to about 20 wt. %, such as from about 1 wt. % to about 17.5 wt. %, such as from about 5 wt. % to about 15 wt. % of a second non-ionic surfactant (e.g., a poloxamer, such as P407), where it has been found that the use of such a combination of skin permeation enhancers can result in a transdermal drug delivery system that exhibits significantly sustained delivery of the API. Because poloxamer-based nonionic surfactants have higher HLB values as described above, they should be used in combination with nonionic surfactants having lower HLB values, such as but not limited to oleth-3, laureth-4, or oleth 5. Further, if a poloxamer-based nonionic surfactant is used, the ratio of the one or more lower HLB value nonionic surfactants having an HLB value of less than about 12 to the poloxamer-based nonionic surfactant or non-ionic surfactant having an HLB value of greater than about 18 should range from about 1.1:1 to about 5:1, such as from about 1.25:1 to about 4:1, such as from about 1.5:1 to about 3:1.

Further, the present inventors have found that the ratio of the API to the skin permeation enhancer impacts the formation of the molecular solid suspension, where a ratio of the API to the skin permeation enhancer that is from about 1:2 to about 1:6, such as from about 1:2.25 to about 1:5, such as about 1:2.5 to about 1:4.5 facilitates formation of a stable, solid API molecular dispersion in the drug in adhesive layer.

g. Other Excipients

The molecular solid suspension drug in adhesive layer can also include gelling agents and/or thickening and/or suspending agents and/or polymers and/or adhesive polymers and/or pressure sensitive adhesive polymers known to those skilled in the art either alone or in combinations thereof without any limitation to following like natural polymers, polysaccharides and its derivatives such as but not limited to (agar, alginic acid and derivatives, cassia tora, collagen, gelatin, gellum gum, guar gum, pectin, potassium, or sodium carrageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxypropyl cellulose, hydroxylpropylmethyl cellulose, hydroxypropyl methylcellulose acetate succinate etc.), synthetic polymers and its derivatives such as without any limitation to carboxyvinyl polymers or carbomers (Carbopol 940, Carbopol 934, Carbopol 971p NF), polyethylene, and its copolymers, clays such as but not limited to (silicates, bentonite), silicon dioxide, polyvinyl alcohol, acrylic polymers (Eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents and/or polymers and/or adhesive polymers and/or pressure sensitive adhesive polymers.

The molecular solid suspension drug in adhesive layer can also include plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing).

The molecular solid suspension drug in adhesive layer can further include solubilizers, additional surfactants, emulsifying agents, dispersing agents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxy1-6-glycerides, lauryl polyoxyl-6-gylcerides, polyglycery1-3-dioleate, diethylene glycol monoethyl ether, propylene glycol monolaurate type I, polyglyceryl-3-dioleate, caprylocaproylpolyoxyl-8 glycerides, cyclodextrins and others.

The molecular solid suspension drug in adhesive layer can further include excipients or chemicals known to those skilled in the art either alone or in combination thereof without any limitation to following like cholecaciferol, vitamin D3, Vitamin B12, cyanocobalamin, Vitamin E, tocopherol, tocopherol acetate, BHA, BHT, propyl gallate, ascorbyl palmitate, and other antioxidants or protectants and or atypical humectants, where such excipients if in solid form are added to the molecular solid suspension before or after the addition of the solubilized drug or API into the system to assist in formation of the suspended particle. For instance, it was found that incorporation of panthenol and/or other humectants into a lenalidomide molecular solid suspension inhibits hydrolytic degradation in long term stability studies held at room temperature and elevated temperatures above 40° C. Such humectants can be solid at room temperature and is soluble to a significant extent within the process solvent of either the API solvent or the solvent of insoluble carrier excipients of the formulation. For example, the humectant can include a sugar, a sugar alcohol, a sugar ester, a polyol, phytantriol, pantothenic acid, urea, tocopherol polyethylene glycol succinate, a polyethylene glycol (PEG) with a molecular weight greater than about 1450, hyaluronic acid, salicylic acid or derivatives thereof, an alpha hydroxy acid or derivative thereof, an amino acid or derivative thereof, panthenol, or a combination thereof. Further, the humectant can present in an amount ranging from about 0.5 wt. % to about 15 wt. %, such as from about 1 wt. % to about 10 wt. %, such as from about 2.5 wt. % to about 7.5 wt. % based on the dry weight of the molecular solid suspension drug in adhesive layer of the transdermal drug delivery system.

### II. Backing Layer

Referring again to FIG. 1, in addition to the molecular solid suspension drug in adhesive layer 110, the transdermal drug delivery system 100 of the present disclosure can include a backing layer 120 that forms the exterior surface 140 of the transdermal drug delivery system 100. The backing layer 120 can be occlusive in nature and can protect the polymer layer (and any other layers present) from the environment and prevents loss of the drug and/or release of other components to the environment during use. Materials suitable for use as backing layers are well-known known in the art and can comprise films of polyester, polyethylene, polypropylene, vinyl acetate resins, ethylene/vinyl acetate copolymers, ethylene/vinyl alcohol copolymers, polyvinyl chloride, polyurethane, cotton, cellulose(s), and the like, in part or in multi-laminate or co-extruded forms, which may include metal foils, aluminum vapor coated plastics, non-woven fabric, cloth and commercially available laminates. A typical backing material has a thickness in the range of 2 to 1000 micrometers. For example, 3M's Scotchpak® 1012 or 9732 (a polyester film with an ethylene vinyl acetate copolymer heat seal layer), 9723 (a laminate of polyethylene and polyester), 9754 (a polyester film backing laminate), or CoTran® 9720 (a polyethylene film), Japan Vilene's polyester films such as EH-1212, or non-woven polyesters, such as EW2080S, EW-9100, EW-9050, EW-2500N, etc., are useful in the transdermal drug delivery systems described herein, as are Dow@backing layer films, such as Dow@BLF 2050 (a multi-layer backing comprising ethylene vinyl acetate layers and an internal SARAN@layer as well as Kuraray's ethylene-vinyl alcohol based films, such as, EF-F, EF-E, EF-XL, VM-XL, and HF-ME.

### III. Release Liner

Referring still to FIG. 1, in addition to the molecular solid suspension drug in adhesive layer 110 and the backing layer 120, the transdermal drug delivery system 100 of the present disclosure can also include a release liner 130 disposed on the skin-contacting surface 150 of the transdermal drug delivery system that can protect the molecular solid suspension drug in adhesive layer 110 of the transdermal drug delivery system 100 until it is ready to be applied to a patient's skin. Once the transdermal drug delivery system 100 is to be applied to a patient's skin at its skin-contacting surface 150, the release liner 130 can be removed and discarded. Materials suitable for use as release liners are well-known known in the art, such as polymers or fluids of silicone or fluorosilicone or fluorocarbon coated or casted or cured onto a substrate, such as polyester, polyethylene (LDPE or HDPE), styrene, polyvinylchloride (PVC) films, which may include but not limited to commercially available products from Dow Corning Corporation designated Bio-Release® liner and Syl-off® 7610, Loparex's PET release liner which are silicone-coated polyester films, Japan Vilene's silicone coated polyester release liners, Saint Gobain's 4130, 4140, 7819, 7748, 7754, 8005, 8310, 6113, 6113A, 7015, 6024, 8312, 8312N, or 9011 liner, and 3M's 1020, 1022, 9741, 9744, 9748, 9749, and 9755 Scotchpak® liners, which are fluoropolymer-coated polyester films.

### IV. Overlay System

In addition to the molecular solid suspension drug in adhesive layer 110, the backing layer 120, and the release liner 130, the transdermal drug delivery system 100 of the present disclosure can also include an optional or inherent overlay system 160 to ensure the transdermal drug delivery system is adhered and secured to the patent's skin during the entire intended wear period. The overlay system 160 can be an occlusive or non-occlusive material made from cloth, fabric, paper, foam, or plastics, and incorporates an adhesive layer for skin contact and adherence. Materials suitable for use as overlay systems are well-known known in the art and include but not limited to the commercially available products of medical tapes, such as 3M's Cotran® 1523, 2480, 2484, 2476P, 9693, 9695, 9699, 9865, 9907T, or 9952. Overlay systems may be customized in a manner to ensure compatibility with the drug-in-adhesive system to mitigate migration or cold flow and ensure adequate interlaminar adhesion to the drug-in-adhesive system and overall for proper adhesion to patient during the intended wear period. In most cases, molecular solid suspensions are formulated in a manner which inhibits the ability of the drug-in-adhesive matrix to act solely alone to adhere to the patient for the intended wear period. In this case, the present inventors have found that the overlay system can serve to adhere the system to the patient, reduces potential for cold flow, and ensure patch is almost 100% adhered to the patient during the wear period which ensures ability for the TDS to deliver consistently and therapeutically.

### V. Method of Making the Transdermal Drug Delivery System

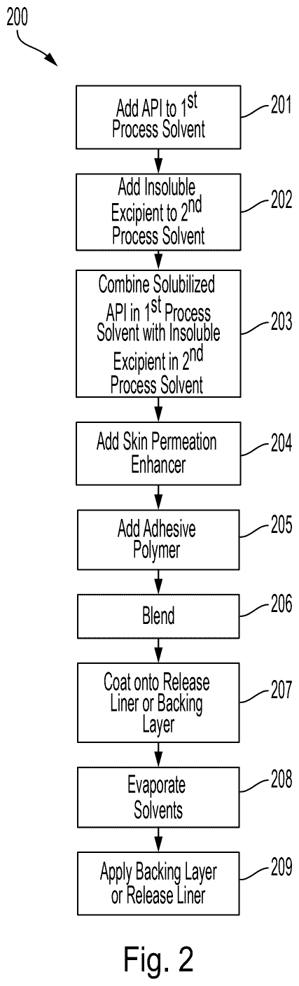

Generally, the molecular solid suspension drug in adhesive layer of the present disclosure is made by combining the components in a specific order, resulting in the ability to form a transdermal drug delivery system with a solid dispersion of the drug in adhesive layer that exhibits improved permeation of the API through the skin. Referring to FIG. 2, one method 200 of making a formulation for a transdermal drug delivery system of the present disclosure is shown. First, in step 201, the API is added to a first process solvent to form a solution where the API is solubilized or dissolved in the solvent to form a true solution. Meanwhile, in step 202, the insoluble carrier excipient is added to a second process solvent to form a dispersion of particles of the insoluble carrier excipient in the second process solvent. Next, in step 203, the solubilized API in the first process solvent can be combined with the insoluble carrier excipient dispersed in the second process solvent, where the components are blended to achieve uniformity. Next, in step 204, the skin permeation enhancer can be added to the mixture, followed by the addition of additional components such as the pressure sensitive adhesive in step 205. Thereafter, in step 206, the aforementioned components are blended or mixed to homogenize and form a milky white uniform suspension. In addition, it is to be understood that a humectant can be added to the second process solvent in step 202 prior to adding the API in step 203, and/or a humectant can be added after step 203 after the API is added, such as before, during, or after steps 204 through 206. Then, in step 207, the resulting molecular solid suspension drug in adhesive layer can be applied to and allowed to dry on a release liner or backing layer on one surface, after which any organic solvents present can be allowed to evaporate in step 608. Then, the opposing surface of the molecular solid suspension drug in adhesive layer can be applied to (e.g., laminated to) a backing layer or release liner in step 209. Thereafter, individual transdermal drug delivery systems can be die-cut from a large sheet of the formed transdermal drug delivery system, either with or without an inherent overlay system that ensures adhesion to a patient, where it is understood that inherent overlay systems are not drug-bearing and can be non-woven/non-occlusive or occlusive in nature.

### VI. Method of Treatment Using the Transdermal Drug Delivery System

Once the transdermal drug delivery system of the present disclosure including molecular solid suspension drug in adhesive layer is made according to the specifications and steps described above, it can be applied a surface of skin (e.g., the strata layer of skin) of a wearer so that the API can be delivered in a controlled manner to the wearer. In some embodiments, prior to applying the transdermal drug delivery system to the surface of skin, the surface of skin can be microneedled with a dermally-applied device (e.g., a derma roller, a stamp, etc.). The device can, in some embodiments, be a derma roller or any other device that includes a plurality of needles. In some embodiments, the derma roller can include needles having a length of from about 0.2 millimeters to about 2.5 millimeters, such as from about 0.3 millimeters to about 2.25 millimeters, such as from about 0.4 millimeters to about 2.0 millimeters. In some embodiments, the needles can have a length ranging from about 0.5 millimeters to about 1.5 millimeters.

The use of a microneedling device such as a derma roller imparts a number of microneedles, typically, 192 or 540 microneedles per derma roller of standard size, to be used for pretreating a surface of skin. Other sizes have been evaluated which include additional microneedling discs across a wider roller, where the 540 microneedle 1.5 cm width roller includes nine (9) discs with sixty (60) microneedles per disc. Another standard roller is referred to as a “body derma roller” with a 3.8 cm width that includes eighteen (18) discs with sixty (60) microneedles per disc for a total of 1080 microneedles per roller.

The number of microneedling penetrations made depends on the number of width passes made over the intended skin conditioning area as it pertains to the actual width of the derma roller. For an active area of 7.1 cm×7.1 cm (˜50 cm²) of the current design for a lenalidomide patch, the derma roller of 3.8 cm width would cover the 7.1 cm width or length with approximately 0.5 cm total overlay (7.6 cm width total). This would serve the purposes of obtaining suitable skin conditioning of the area to which the patch is to be applied from a two (2) width pass perspective.

The number of microneedling penetrations also depends on the number of back and forth passes on a skin conditioning area or surface of skin to be treated with proper instructions for use. In one embodiment, the representative number of microneedle penetrations per surface area from a single pass in one direction can be about 57 microneedles per cm², such as when the number of microneedles per derma roller is 540 microneedles, and the roller is 1.5 cm wide by 6.28 cm in circumference, providing a microneedling area of 9.4 cm². In another embodiment, the representative number of microneedle penetrations per surface area from a single pass in one direction can be about 45 microneedles per cm², such as when the number of microneedles per derma roller is 1080 microneedles, and the roller is 3.8 cm wide by 6.28 cm in circumference, providing a microneedling area of 23.9 cm².

Further, it should be understood that, based on the size of the derma roller used and the surface area of skin to be treated, multiple passes can be employed, and Tables 6-8 summarize various embodiments that are contemplated by the present disclosure.

With the basic dimensions of the measured penetration of approximately 50 μm×100 μm per microneedle, the percent (%) coverage of the applied area (e.g., surface of skin to be treated) can be ascertained as follows. In each of the scenarios described above, no more than 10% coverage of the applied surface area for derma rolling is expected to be conditioned, as shown in Tables 6-8 below. Without intending to be limited by any particular theory, the impact on the level of skin conditioning due to more derma rolling to increase percent coverage will likely lead to ever increasing skin permeation, however, at the probability of increased adverse events, such as, skin irritation.

TABLE 6
Pore Size of Microneedles

| Estimated Pore | Pore Size | Microneedle | % Coverage on |
|---|---|---|---|
| Dimensions | as Area | Pores per cm² | per cm² basis |
| 50 μm by 100 μm | 5,000 cm² | 20,000 | 100% |

TABLE 7
540 Microneedle Derma Roller % Coverage

| # of | | | | # of Micro- | |
|---|---|---|---|---|---|
| Micro- | | One Pass | # of | needles | % Coverage |
| needles/ | # of | Front | Geometric | Applied | on per cm² |
| cm² | Passes | and Back | Dimensions | per cm² | Basis |
| 57 | 1 | 1 | 1 | 57 | 0.3% |
| 57 | 1 | 2 | 2 | 229 | 1.2% |
| 57 | 2 | 2 | 2 | 458 | 2.3% |
| 57 | 4 | 2 | 2 | 917 | 4.6% |
| 57 | 4 | 2 | 3 | 1368 | 6.8% |
| 57 | 4 | 2 | 4 | 1824 | 9.1% |

TABLE 8
1080 Microneedle Derma Roller % Coverage

| # of | | | | # of Micro- | |
|---|---|---|---|---|---|
| Micro- | | One Pass | # of | needles | % Coverage |
| needles/ | # of | Front | Geometric | Applied | on per cm² |
| cm² | Passes | and Back | Dimensions | per cm² | Basis |
| 45 | 1 | 1 | 1 | 45 | 0.2% |
| 45 | 1 | 2 | 2 | 180 | 0.9% |
| 45 | 2 | 2 | 2 | 360 | 1.8% |
| 45 | 4 | 2 | 2 | 720 | 3.6% |
| 45 | 4 | 2 | 3 | 1080 | 5.4% |
| 45 | 4 | 2 | 4 | 1440 | 7.2% |

After the surface of skin is microneedled, the transdermal drug delivery system 100 of the present disclosure can be applied to the surface of skin by removing the release liner 130 so that a skin contacting surface 150 of the molecular solid suspension drug in adhesive layer 110 is exposed. The skin contacting surface 150 can then be positioned on the now microneedled surface of skin. Further, the transdermal drug delivery system 100 can be held in place with an overlay system 160 as described above.

The present disclosure may be better understood by reference to the following examples.

### Examples Overview

Efforts were pursued to understand solubility of the various APIs in various organic solvents and excipients typically used in pharmaceutical API products, and specifically, in transdermal formulations. It was found that NMP or DMSO has significant affinity for APIs having low water solubility, which exceeds all other tested materials by a factor of more than 2-fold of the observed solubility at room temperature as compared to other organic solvents of these types of systems, such as ethyl acetate, heptane, isopropyl alcohol, ethanol, or toluene, where it was found that the selected solvent, such as NMP or DMSO, can solubilize up to about 20 wt. % or more of the API for ease of incorporation into the drug in adhesive layer. The key aspect of the solvent is that it should be miscible with other components, yet volatile in nature at elevated temperatures during the curing process to achieve ICH Residual Solvent limits after processing. Of particular interest are the polar aprotic solvents; however, it should be understood that other solvents may be suitable. Another key aspect of the solvent system is such that the API is forced out of solution to form particulates upon addition to the remaining components of the blend including, but not limited to, the adhesive polymer, insoluble carrier excipients, other excipients, skin permeation enhancers, and/or other process solvents.

The formulation strategy was to prepare a monolayer or monolithic drug in adhesive layer coated between a backing layer and a disposable release liner. Surprisingly, it was found that the addition of API in a solubilized form in a first process solvent was necessary to incorporate the API into a solution for addition and to produce a consistent and uniform polymer blend and resulting laminate after the evaporation of process solvents including the first process solvent of the solubilized API, which resulted in a molecular solid suspension of the API, where the API forms a particulate with the other excipients and is suspended uniformly within the polymer blend with the solvent system during the blending process to produce a homogeneous wet suspension, and the API particulates are formed in situ in the blend prior to coating and evaporation of the solvent system.

A micronized grade of insoluble carrier excipient, such as crospovidone, a cross-linked povidone, was incorporated to allow molecular adsorption of the API onto a solid porous substrate and dispersion. Other substrates, such as those mentioned above for insoluble materials to act as the substrate, may be viable such that they disperse within the drug in adhesive layer or matrix and allow for affinity of crystalline and/or molecular dispersion of the API.

Initial formulations, as shown in Examples 1-4, were made to assess molecular solid suspension drug in adhesive layers with compositions in the same acrylic pressure sensitive adhesive (Durotak 87-4098), a non-functional acrylic copolymer with vinyl acetate, as well to evaluate the matrix in the presence of (1) an insoluble carrier excipient (crospovidone) and (2) the insoluble carrier excipient (crospovidone) and a permeation enhancer (a nonionic surfactant of the fatty alcohol derivatives, Brij L4, or Laureth-4), where it is to be understood that the components used can be replaced with any of the above-mentioned excipients of similar nature and properties, such as HLB value, particle size, molecular weight, melting temperature, Log P value, and other physicochemical properties.

For in vitro permeation testing, the prepared transdermal formulations in the following examples were subjected to a flux (in vitro permeation) test as follows with either full-split thickness human cadaver skin or Strat-M® synthetic membrane. The full-split thickness human cadaver skin is prepared by thawing at room temperature in DI water, after which it is placed into a 55° C. water bath for a few minutes. After removal from elevated temperature water bath, the stratum corneum layer is removed, inspected, blotted, and cut into suitable sized samples for placement on a modified Franz vertical diffusion cell. Meanwhile, the Strat-M® synthetic membrane is utilized as instructed by the Millipore-Sigma instruction guide on its use. The patches are die cut to a 5/16″ diameter (˜0.5 cm²) and affixed to the skin with use of an overlay to secure patch to skin. Receiver media is either standard saline (0.9% NaCl in DI water) or a fixed concentration (1%-5%) of povidone in DI Water. Sample volume is 5 mL, Franz cell volume is 5 mL. Sampling intervals are over the intended wear period from 0 to up to 168 hours. Sample time points are typically at 8 hours, 24, 48, 72, 96, 120, 144 and 168 hours. Actual patch size is 0.5 cm² with an effective flux area of about 0.6 cm². Aliquots taken are analyzed by HPLC method for each independent API. Typically, 3-5 replicates per formulation are performed to ensure adequate statistical significance is obtained for each study.

### Example 1

Example 1 focuses on the development of a molecular solid suspension drug in adhesive formulation for antipsychotics or tricyclics (e.g., olanzapine). The formulations tested are shown below in Table 9.

TABLE 9
Olanzapine Formulations

| Excipients | RDNB-0011-162-1 | | RDNB-0011-162-2 | | RDNB-0011-162-3 | |
|---|---|---|---|---|---|---|
| Olanzapine | 5 | wt. % | 5 | wt. % | 5 | wt. % |
| Crospovidone | 0 | wt. % | 5 | wt. % | 5 | wt. % |
| Laureth-4 | 0 | wt. % | 0 | wt. % | 20 | wt. % |
| Durotak 87-4098 | 95 | wt. % | 90 | wt. % | 70 | wt. % |

| | | | |
|---|---|---|---|
| OBSERVATIONS | Dispersion with | Dispersion with | Dispersion with |
| | uniformity with | uniformity with | uniformity with |
| | some larger white | some larger white | some larger white |
| | dispersed particles | dispersed particles | dispersed particles |

Process solvents include n-methyl-2-pyrrolidone for solubilizing API and ethyl acetate for other excipients

### Example 2

Example 2 focuses on the development of a molecular solid suspension drug in adhesive formulation for BTK inhibitors (e.g., ibrutinib). The formulations tested are shown below in Table 10.

TABLE 10
Ibrutinib Formulations

| Excipients | RDNB-0011-164-1 | | RDNB-0011-164-2 | | RDNB-0011-164-3 | |
|---|---|---|---|---|---|---|
| Ibrutinib | 5 | wt. % | 5 | wt. % | 5 | wt. % |
| Crospovidone | 0 | wt. % | 5 | wt. % | 5 | wt. % |
| Laureth-4 | 0 | wt. % | 0 | wt. % | 20 | wt. % |
| Durotak 87-4098 | 95 | wt. % | 90 | wt. % | 70 | wt. % |

| | | | |
|---|---|---|---|
| OBSERVATIONS | Dispersion with | Dispersion with | Dispersion with |
| | uniformity with | uniformity with | uniformity with |
| | some larger white | some larger white | some larger white |
| | dispersed particles | dispersed particles | dispersed particles |

Process solvents include n-methyl-2-pyrrolidone for solubilizing API and ethyl acetate for other excipients

### Example 3

Example 3 focuses on the development of a molecular solid suspension drug in adhesive formulation for steroids (e.g., dexamethasone). The formulations tested are shown below in Table 11.

TABLE 11
Dexamethasone Formulations

| Excipients | RDNB-0011-171-1 | | RDNB-0011-171-2 | | RDNB-0011-171-3 | |
|---|---|---|---|---|---|---|
| Dexamethasone | 5 | wt. % | 5 | wt. % | 5 | wt. % |
| Crospovidone | 0 | wt. % | 5 | wt. % | 5 | wt. % |
| Laureth-4 | 0 | wt. % | 0 | wt. % | 20 | wt. % |
| Durotak 87-4098 | 95 | wt. % | 90 | wt. % | 70 | wt. % |

| | | | |
|---|---|---|---|
| OBSERVATIONS | Dispersion with | Dispersion with | Dispersion with |
| | uniformity with | uniformity with | uniformity with |
| | some larger white | some larger white | some larger white |
| | dispersed particles | dispersed particles | dispersed particles |

Process solvents include dimethyl sulfoxide for solubilizing API and ethyl acetate for other excipients

### Example 4

Example 4 focuses on the development of a molecular solid suspension drug in adhesive formulation for immunomodulatory agents (e.g., lenalidomide). The formulations tested are shown below in Table 12:

TABLE 12
Lenalidomide Formulations

| Excipients | RDNB-0011-165-1 | | RDNB-0011-165-2 | | RDNB-0011-165-3 | |
|---|---|---|---|---|---|---|
| Lenalidomide | 5 | wt. % | 5 | wt. % | 5 | wt. % |
| Crospovidone | 0 | wt. % | 5 | wt. % | 5 | wt. % |
| Laureth-4 | 0 | wt. % | 0 | wt. % | 20 | wt. % |
| Durotak 87-4098 | 95 | wt. % | 90 | wt. % | 70 | wt. % |

| | | | |
|---|---|---|---|
| OBSERVATIONS | Dispersion with | Dispersion with | Dispersion with |
| | uniformity with | uniformity with | uniformity with |
| | some larger white | some larger white | some larger white |
| | dispersed particles | dispersed particles | dispersed particles |

Process solvents include n-methyl-2-pyrrolidone for solubilizing API and ethyl acetate for other excipients

From the above formulations, it was found that the order of addition of the excipients/components was important to maintain consistency in formulation outcome, i.e., homogeneous blending. The optimized order of addition includes dissolving the API in the presence of a first process solvent (e.g., NMP or dimethyl sulfoxide) as a premix. In a separate container, crospovidone (e.g., Kollidon CLM) was dispersed in a second process solvent, such as ethyl acetate, as appropriate for the formulation. Next, the API premix was added to the solvent dispersed crospovidone. A dispersion is then formed, and mixing should be performed to achieve uniformity. Then, other excipients are added, such as the skin permeation enhancer, laureth-4. The resulting formulation is then blended or mixed to achieve uniformity. Next, other excipients, including the adhesive polymer, are added and blended to homogenize the formulation and to form a milky white uniform suspension.

The invention provides a transdermal delivery system (TDS) for administration of API comprising: an active substance area comprising a pharmaceutical composition of at least one (1) API, at least one (1) insoluble carrier excipient to act as particle substrate; at least one (1) excipient to act as permeation enhancer, solubilizer, or plasticizer; and at least one (1) polymer to act as an adhesive backbone, such as acrylic pressure sensitive adhesive, including polyisobutylene, silicone PSAs, or combinations thereof. The cured adhesive composition is coated between an impermeable backing layer and a release liner. The composition may incorporate an overlay adhesive system.

The formulations as described above can provide a pharmaceutical composition for the transdermal delivery of the API for up to 7 days.

As discussed above, the API can be present in an amount such as from about 3 wt. % to about 30 wt. %, or even higher, and this presence within the system is at a significantly higher level than the system has ability to dissolve or maintain at supersaturated levels, such that the API is incorporated at a level well above saturation or even supersaturation. This level is referred to as the oversaturation level, which is about 1.2×, 1.3×, 1.4×, 1.5×, 2×, 3×, 4×, 5×, 10× or up to about 50× the capacity of the system to solubilize the API, such that when producing the dispersion in situ within the blending process, the blend remains a uniform and homogeneous dispersion of similar particle sizes amenable to a coating process and without settling of the API during the coating process. Crystallization can occur with seed crystals or particulates within a supersaturated system. However, in the system of the current disclosure, an oversaturated system does not crystallize in the presence of seed crystals or particles but maintains a uniform particulate dispersion or suspension of particulates within the substrate or drug-in-adhesive layer.

The above formulations in Tables 4-7 demonstrate the utility of the current disclosure across a multitude of API drug types, with the general approach being to incorporate an oversaturated amount of API into the formulation.

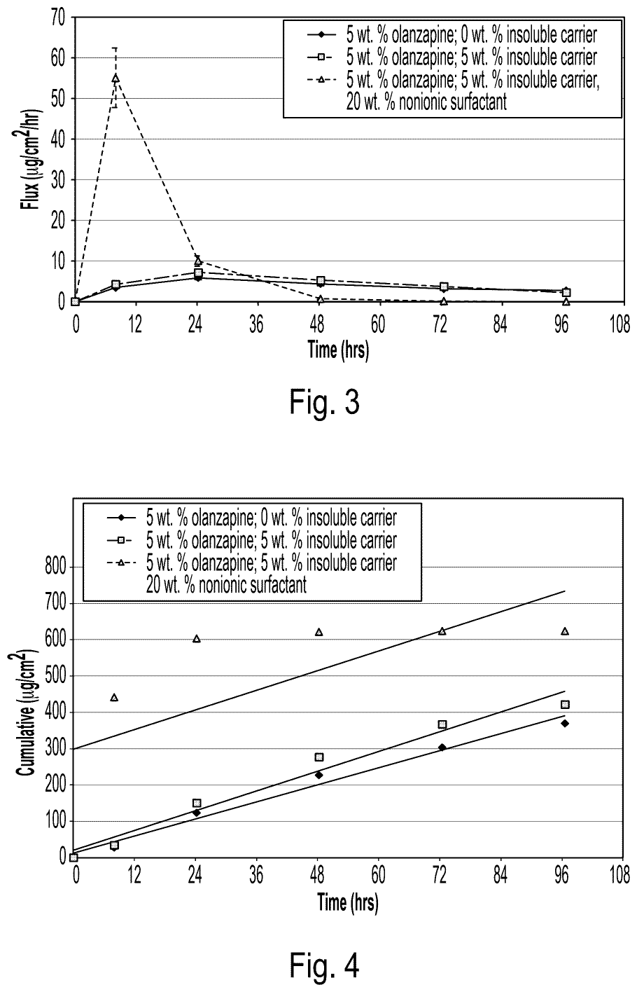

In the current disclosure, it was found that applicability encompasses a vast range of molecular compounds and is not strictly in the immunomodulatory imide class of compounds. The applicability was found to rationalize around the physicochemical characteristics of molecules based on melting points and water solubilities as discussed above. Additional data regarding the flux of the API through the skin is shown below with respect to Examples 5-8 and FIGS. 3-10.

### Example 5

Referring to FIGS. 3-4, the present disclosure further provides an exemplary formulation including 5 wt. % olanzapine, 5 wt. % of an insoluble carrier (e.g., Kollidon CLM), 20 wt. % of a nonionic surfactant (e.g., Laureth-4), and 70 wt. % of an acrylic pressure sensitive adhesive (e.g., Durotak 87-4098) that was compared to 5 wt. % olanzapine and 95 wt. % of a pressure sensitive adhesive (e.g., Durotak 87-4098) and 5 wt. % olanzapine, 5 wt. % of an insoluble carrier (e.g., Kollidon CLM), and 90 wt. % of an acrylic pressure sensitive adhesive (e.g., Durotak 87-4098). Superior performance is shown with the molecular solid suspension with an approximately 1.2× increase in slope for cumulative flux (FIG. 4). However, performance of the system is limited by the drug loading of the system. For olanzapine, it is surmised that saturation of the system is obtained close to that of the adhesive used in this case, and the significance of the system of the molecular solid suspension is the overwhelming amount of increase in availability at the about 8-hour time point with an increase of about 16×, as shown in FIG. 3 for average flux. In this case, higher drug loading is necessary to achieve the sustainability of the delivery profile at such a higher rate, where 100% depletion was observed after about 24 hours compared to 60% depletion in other two (2) formulations over 72 hours, and therefore due to differences in delivery profile, slopes of these are not comparable over the full delivery period. The current disclosure suggests that a significantly higher rate of delivery is achievable.

### Example 6

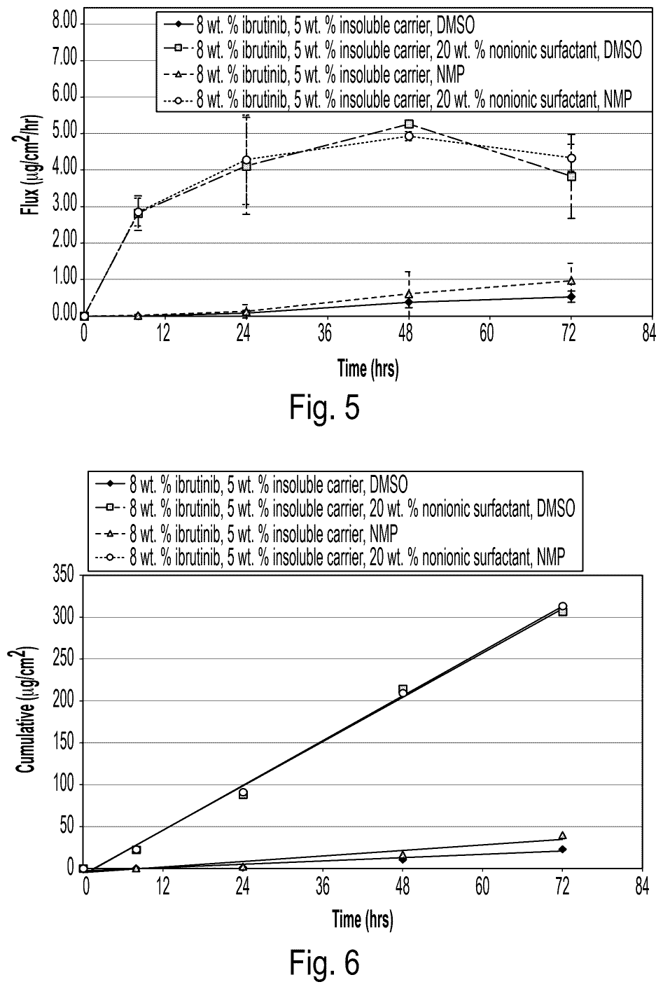

Referring to FIGS. 5-6, the present disclosure further provides an exemplary formulation of a molecular solid suspension that includes 8 wt. % ibrutinib, 5 wt. % of an insoluble carrier (e.g., Kollidon CLM), 20 wt. % of a nonionic surfactant (e.g., Laureth-4), and 67 wt. % of an acrylic pressure sensitive adhesive (e.g., Durotak 87-4098) with either NMP or DMSO as the process solvent. The superior performance of these formulations in terms of average flux and cumulative flux is shown in FIGS. 5-6 in which the formulations are compared to 8 wt. % ibrutinib, 5 wt. % of an insoluble carrier (e.g., Kollidon CLM) and 87 wt. % of an acrylic pressure sensitive adhesive (e.g., Durotak 87-4098) with either NMP or DMSO as the process solvent, where the superior performance with the skin permeation enhancer is evidenced by a greater than about 10× increase in slope.

### Example 7

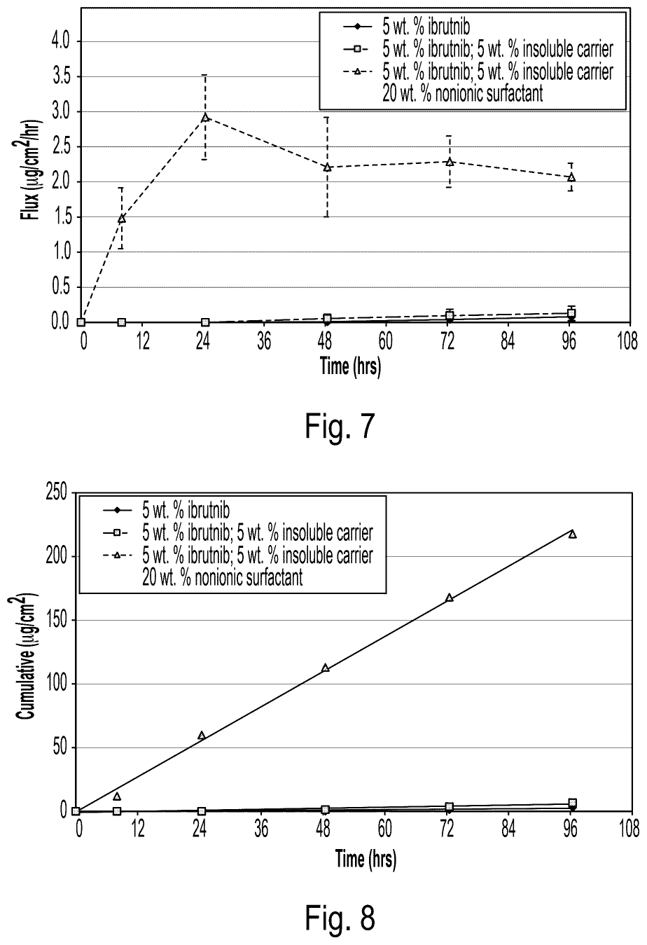

Referring to FIGS. 7-8, the present disclosure further provides an exemplary formulation of a molecular solid suspension that includes 5 wt. % ibrutinib, 5 wt. % of an insoluble carrier (e.g., Kollidon CLM), 20 wt. % of a nonionic surfactant (e.g., Laureth-4), and 70 wt. % of an acrylic pressure sensitive adhesive (e.g., Durotak 87-4098). The superior performance of this formulation in terms of average flux and cumulative flux is shown in FIGS. 7-8 in which the formulation is compared to 5 wt. % ibrutinib, 5 wt. % of an insoluble carrier (e.g., Kollidon CLM) and 90 wt. % of a pressure sensitive adhesive (e.g., Durotak 87-4098) and 5 wt. % ibrutinib and 95 wt. % of an acrylic pressure sensitive adhesive (e.g., Durotak 87-4098). The molecular solid suspension exhibits approximately a 30× increase in slope.

### Example 8

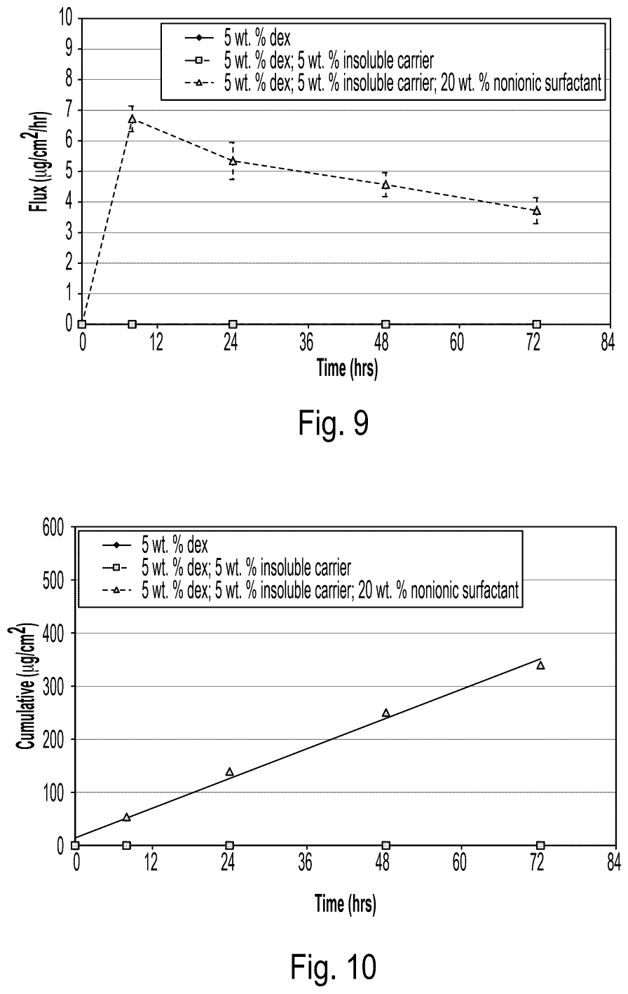

Referring to FIGS. 9-10, the present disclosure further provides an exemplary formulation of a molecular solid suspension that includes 5 wt. % dexamethasone, 5 wt. % of an insoluble carrier (e.g., Kollidon CLM), 20 wt. % of a nonionic surfactant (e.g., Laureth-4), and 70 wt. % of an acrylic pressure sensitive adhesive (e.g., Durotak 87-4098). The superior performance of this formulation in terms of average flux and cumulative flux is shown in FIGS. 9-10 in which the formulation is compared to 5 wt. % dexamethasone, 5 wt. % of an insoluble carrier (e.g., Kollidon CLM) and 90 wt. % of an acrylic pressure sensitive adhesive (e.g., Durotak 87-4098) and 5 wt. % dexamethasone and 95 wt. % of an acrylic pressure sensitive adhesive (e.g., Durotak 87-4098). The molecular solid suspension exhibits almost an infinite increase in slope.

### Example 9

In Example 9, the effect of the incorporation of a humectant (e.g., panthenol) into the molecular solid suspension on the API stability and permeability is shown. It has been observed that the TO release testing of similar formulations with Lenalidomide exhibited significantly different degradation of the drug products freshly made, in which, it is shown that panthenol exhibits hydrolytic production of the API, lenalidomide, which is susceptible to both oxidation and hydrolytic degradation. In the tables below, TDS-009 includes 8 wt. % LLD, 5 wt. % Panthenol, 10% Poloxamer P407, 5 wt. % crospovidone and 22.5 wt. % Brij L4 in the presence of 49.5 wt. % DuroTak 87-4098 acrylic PSA, while TDS-011 is the same as TDS-009 without presence of 5 wt. % Panthenol, making up for the 5 wt. % difference with additional DuroTak 87-4098 acrylic PSA for a total of 54.5 wt. %. As shown below in Tables 13 and 14, the inclusion of the panthenol in TDS-009 results in improved stability (e.g., reduced hydrolytic degradation) and less total impurities compared to TDS-011, which does not include panthenol, during relative retention time testing using liquid chromatography.

TABLE 13
Summary of Test Results (Freshly Made Drug Product)

| | Potency | | | | | |
|---|---|---|---|---|---|---|
| Formula | (% LC) | RRT 0.70 | RRT 0.88 | RRT 1.30 | RRT 1.35 | RRT 1.39 |
| TDS-009 | 98.9% | N/D | 0.05% | 0.16% | N/D | N/D |
| TDS-011 | 93.7% | 0.11% | 0.43% | N/D | N/D | N/D |

| Primary Cause | Hydrolysis | Hydrolysis | Oxidation of | Oxidation of | Oxidation |
|---|---|---|---|---|---|
| | of LLD | of LLD | Panthenol | LLD | of LLD |

TABLE 14
Summary of Test Results (Total Impurities by Degradation Type)

| | Total Hydrolytic | Total Oxidative | |
|---|---|---|---|
| Formula | Degradation | Degradation | Total Impurities |
| TDS-009 | 0.05% | 0.16% | 0.21% |
| TDS-011 | 0.54% | 0.00% | 0.54% |

The historical nature of working with lenalidomide would suggest that the hydrolysis products will continue to grow with time and temperature/relative humidity conditions at a relative rate. The work shown below in Table 15 for 1 year data with a molecular solid suspension of lenalidomide (LLD) n the presence of a humectant such as panthenol suggests that hydrolysis is completely mitigated by its incorporation, where 3-74-1 includes Brij O5, Poloxamer P407, crospovidone, and DuroTak 87-9301 with 5 wt. % panthenol; 3-75-1, 3-75-2, and 3-75-3 include Brij O5, crospovidone, and DuroTak 87-9301 with 1 wt. %, 2 wt. %, and 4 wt. % panthenol without Poloxamer P407, respectively; and 3-75-4, 3-75-5, and 3-75-6 include Brij 05, Poloxamer P407, crospovidone, and DuroTak 87-9301 with 1 wt. %, 2 wt. %, or 4 wt. % panthenol, respectively. Generally, the incorporation of panthenol into the molecular solid suspension results in a 10-fold reduction in hydrolytic degradation.

TABLE 15
Summary of Test Results Aged 1 Year
at Room Temperature (21° C.)

| | Panthenol | | | | |
|---|---|---|---|---|---|
| Formula | wt. % | RRT 0.70 | RRT 0.88 | RRT 1.30 | RRT 1.39 |
| 3-74-1 | 5% | N/D | 0.11% | 0.36% | 0.06% |
| 3-75-1 | 1% | N/D | N/D | N/D | 0.05% |
| 3-75-2 | 2% | N/D | N/D | <0.05% | 0.05% |
| 3-75-3 | 4% | N/D | N/D | 0.13% | 0.06% |
| 3-75-4 | 1% | N/D | N/D | N/D | 0.06% |
| 3-75-5 | 2% | N/D | N/D | 0.05% | <0.05% |
| 3-75-6 | 4% | <0.05% | <0.05% | 0.20% | <0.05% |

| | | | | |
|---|---|---|---|---|
| Primary Cause | Hydrolysis | Hydrolysis | Oxidation of | Oxidation |
| | of LLD | of LLD | Panthenol | of LLD |

This further supports the fact that a drug which is amenable to hydrolysis shows no evidence of hydrolysis in the presence of a humectant such as panthenol, which is not expected to be a hydrolytic protective agent as it oxidizes and would have antioxidant properties such as BHA or BHT. Indeed, limited oxidative degradation is exhibited as well over 1 year drug product stability without significant evidence of API degradation through either primary pathway. Each of the above formulations was protected from light, but not from ambient storage conditions of about 21° C. and ambient relative humidity.

Through an oxidative pathway, panthenol enzymatically cleaves to form pantothenic acid (Vitamin B5) in the human body metabolism of panthenol. This would provide the expected pathway of oxidation to occur in place of lenalidomide oxidation, such that, similar to BHA or BHT, these antioxidants sacrifice themselves to the presence of oxygen within a sealed system and thus elicit antioxidant protective behavior by reacting to the oxygen present before the API has the ability to react. Hydrolysis protection was not expected to occur.

In addition to the protective nature of panthenol, it is expected that the humectant nature of panthenol adds to the performance characteristics of panthenol within the transdermal patch formulations of the present disclosure. This is likely due to the similarity in Log P to lenalidomide and other APIs, where panthenol has a Log P value of −0.9, and the molecular nature of panthenol, comprising a second amine and three primary alcohols which lend itself to a “like-dissolves-like” philosophy as a solid material and not as a liquid solvent-based system of dissolution. It is also surmised that the lower melting point of panthenol (about 66° C. to about 69° C.) offers in a solid system the ability to form a eutectic point with APIs to offer the ability to achieve availability to solubilize and permeate the API from the patch through a semi-permeable membrane, such as human skin.

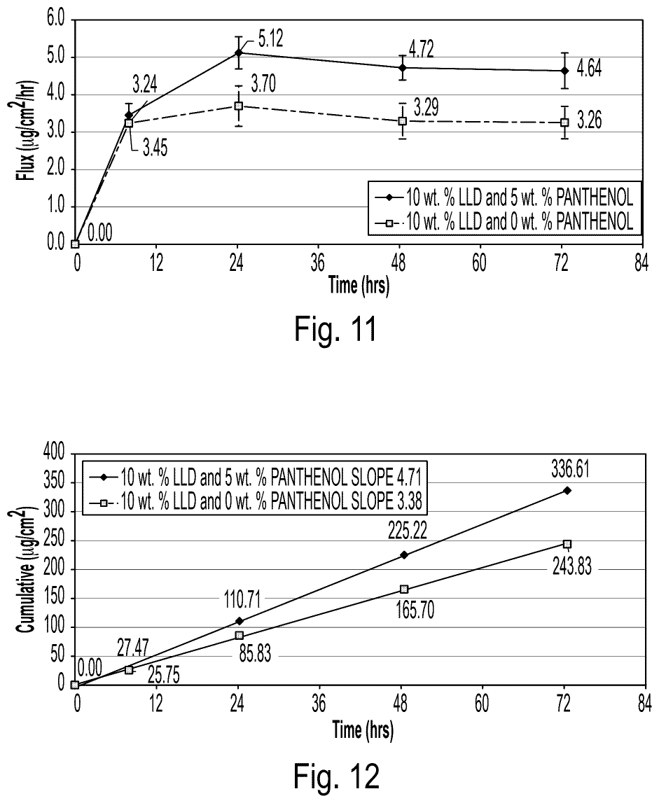

The following study, as summarized in FIGS. 11-16, was completed to support the in vitro permeation of lenalidomide in presence of various concentrations of panthenol to support its use. In some instances, the Log P takes precedence over melting point, in some cases, melting point takes precedence over Log P and in most cases, it is a combination of both attributes which contribute to the availability of the API to release from a molecular solid suspension.

Referring to FIGS. 11-12, the overall permeation of lenalidomide in a molecular solid suspension containing 10 wt. % lenalidomide and 5 wt. % panthenol is about 1.4 times that of the same formulation but excluding panthenol in terms of average flux rate, where the average flux rate for the panthenol-containing formulation was 4.7 μg/cm²/hr after about the 8 hour time point compared to the same drug concentration without panthenol, where the average flux rate was observed to be 3.4 μg/cm²/hr.

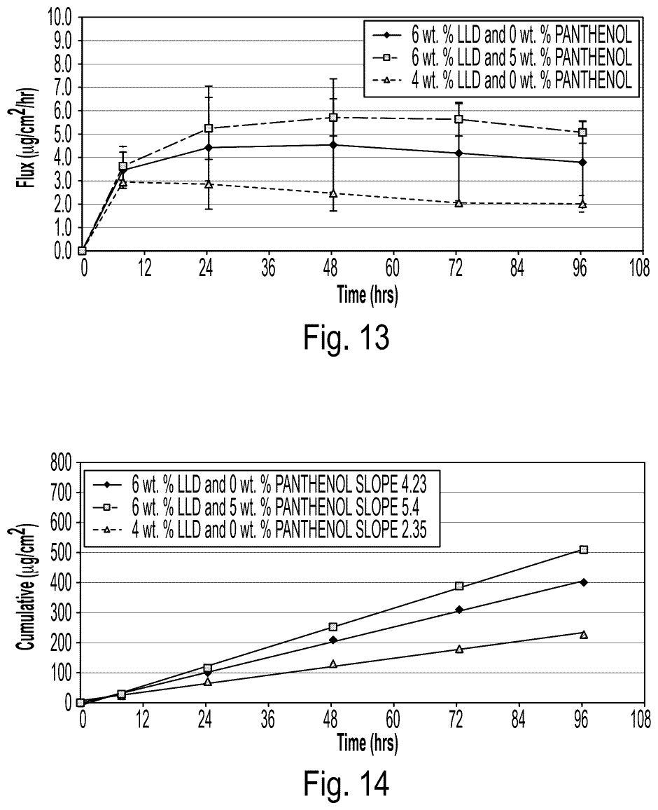

Referring to FIGS. 13-14, the overall permeation of lenalidomide in a molecular solid suspension containing 6 wt. % lenalidomide and 5 wt. % panthenol is about 1.2 times that of the same formulation but excluding panthenol in terms of average flux rate, where the average flux rate for the panthenol-containing formulation was 5.4 μg/cm²/hr after about the 8 hour time point compared to the same drug concentration without panthenol, where the average flux rate was observed to be 4.2 μg/cm²/hr. Where a 4 wt. % drug formulation was incorporated to understand potential saturation or over/under saturation levels, there was a significant decrease in the observed flux rate, which was 2.4 μg/cm²/hr, illustrating that even though the molecular solid suspension still exists within the system, the level of drug must be significantly above the saturated level of solubilized drug to overcome the barrier to permeation and availability of the API.

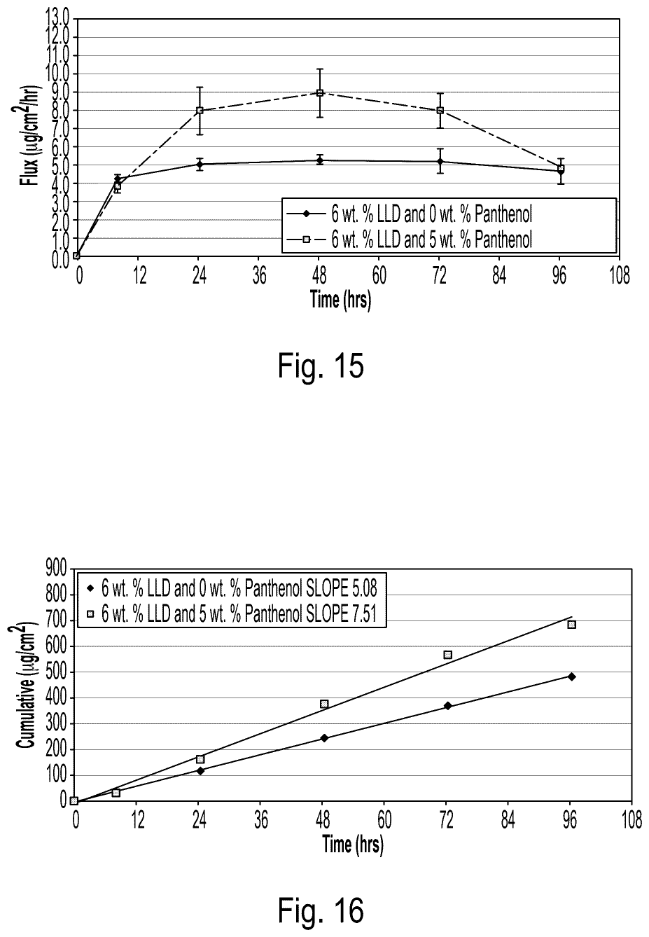

Referring to FIGS. 15-16, the overall permeation of lenalidomide in a molecular solid suspension containing 6 wt. % lenalidomide and 5 wt. % panthenol is about 1.5 times that of the same formulation but excluding panthenol, where the average flux rate was observed at 7.5 μg/cm²/hr after about the 8 hour time point compared to the same drug concentration without panthenol, where the average flux rate was observed to be 5.1 μg/cm²/hr. This is additional confirmation demonstrating that formulations with panthenol exhibit significantly higher availability of API and in turn significant permeability increases compared to formulations not containing panthenol. Thus, the present inventors have found that incorporating a humectant such as panthenol and/or optimizing the API concentration in a molecular solid suspension can increase the permeability of the API.

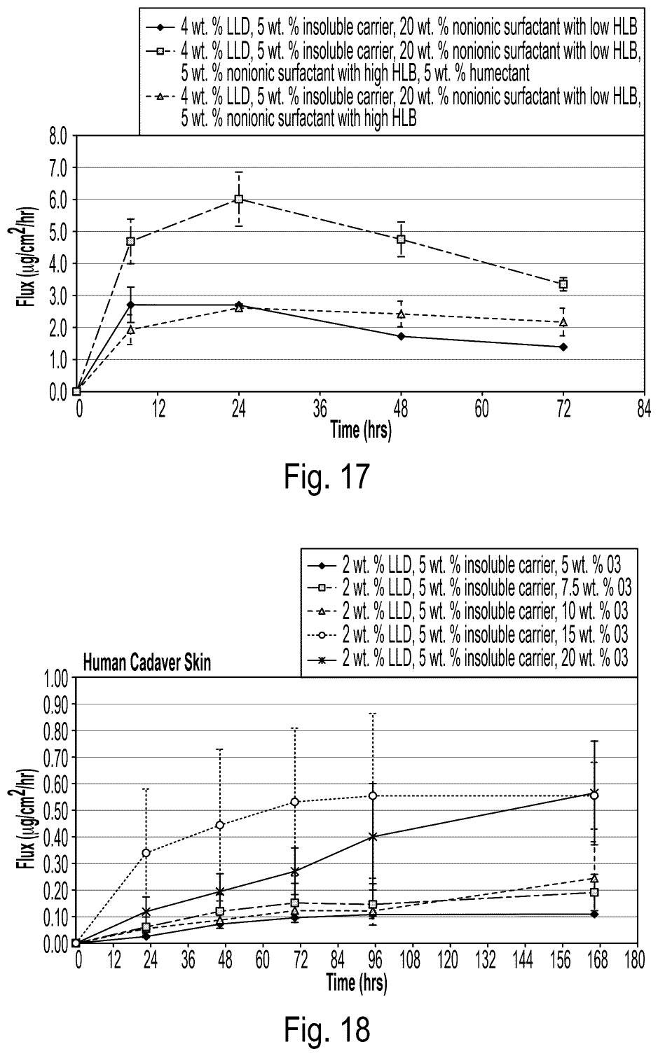

Referring to FIG. 17, additional data showing the impact of a humectant (e.g., panthenol) on the permeation of lenalidomide in a molecular solid suspension is shown. Specifically, FIG. 17 is a graph describing the average flux (micrograms/square centimeter/hour) of lenalidomide with and without a humectant (e.g., panthenol) through Strat-M® (synthetic membrane) for various transdermal drug delivery system formulations that include a nonionic surfactant having a low hydrophilic to lipophilic balance (HLB) value below 12 (e.g., O3) with and without a nonionic surfactant having a high HLB value greater than 12 (e.g., P407, with an HLB of 18-23). As shown, the addition of a humectant (e.g., panthenol) and a high HLB nonionic surfactant (e.g., P407) in combination greatly increases the permeation of lenalidomide compared to when no humectant (e.g., panthenol) is added and to when a humectant (e.g., panthenol) but no high HLB nonionic surfactant (e.g., P407) is added. For instance, at 24 hours, the average flux (micrograms/square centimeter/hour) of rate was observed to be about 6 μg/cm²/hr with both the humectant and high HLB nonionic surfactant (e.g., panthenol and P407) compared to between 2 and 3 μg/cm²/hr when just the humectant (e.g., panthenol) or just the high HLB nonionic surfactant (e.g., P407) was added to the lenalidomide formulation.

### Example 10

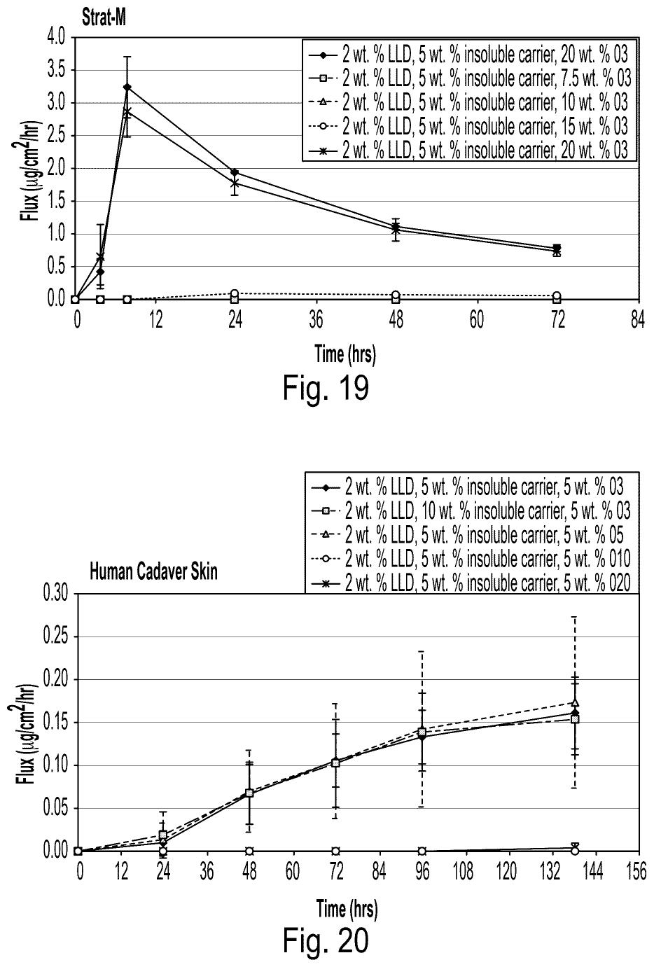

In FIGS. 18-19, the effect of varying the concentration of a lower HLB value (less than 12) non-ionic surfactant from 7.5 wt. % to 20 wt. % was determined for both human cadaver skin and Strat-M® (synthetic membrane) in multiple lenalidomide molecular solid suspensions was demonstrated. For instance, FIG. 18 is a graph describing the average flux (micrograms/square centimeter/hour) of lenalidomide with varying concentrations of a lower HLB value (less than 12) nonionic surfactant through human cadaver skin for various transdermal drug delivery system formulations, while FIG. 19 is a graph describing the average flux (micrograms/square centimeter/hour) of lenalidomide with varying concentrations of a lower HLB value (less than 12) nonionic surfactant through Strat-M® (synthetic membrane) for various transdermal drug delivery system formulations. As shown, the average flux was significantly higher for the formulations containing greater than about 15 wt. % of the lower HLB value nonionic surfactant O3 compared to the formulations containing about 15 wt. % or less of the lower HLB value nonionic surfactant O3.

### Example 11

In FIG. 20, the effect of the HLB value of various non-ionic surfactants on the flux of lenalidomide molecular solid suspensions was demonstrated. As shown in FIG. 20, the average flux (micrograms/square centimeter/hour) of lenalidomide through human cadaver skin for various transdermal drug delivery system formulations that include nonionic surfactants having hydrophilic to lipophilic balance (HLB) values below 12 (e.g., O3 and O5) was increased compared to that of formulations including nonionic surfactants having an HLB value above 12 (e.g., O10 and O20).

### Example 12

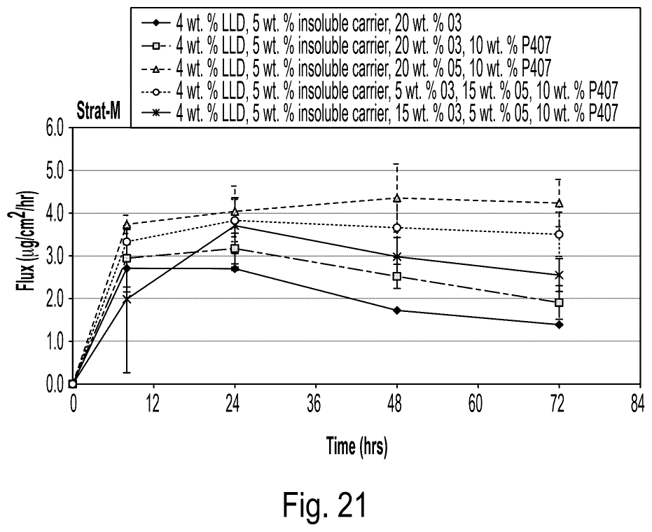

In FIG. 21, the effect of the inclusion of a poloxamer-based nonionic surfactant having an HLB value greater than 12 is demonstrated when used in combination with one or more nonionic surfactants having a lower HLB value less than 12 for various lenalidomide molecular solid suspensions was demonstrated. In particular, FIG. 21 shows that the average flux (micrograms/square centimeter/hour) of lenalidomide through Strat-M® (synthetic membrane) for various transdermal drug delivery system formulations that include nonionic surfactants having hydrophilic to lipophilic balance (HLB) values below 12 (e.g., O3 and O5 both alone and in combination) with a poloxamer-based nonionic surfactant having a higher HLB value greater than 15 (e.g., P407, with an HLB of 18-23) is increased compared to when a poloxamer-based nonionic surfactant is not included.

### Example 13

In Example 13, the effect of using various derma rollers with different needle lengths on a surface of skin to be treated prior to applying the transdermal drug delivery systems of the present disclosure onto the surface of skin on the average flux (micrograms/square centimeter/hour) and cumulative permeation (micrograms/square centimeter) of the molecular solid state suspensions contemplated by the present disclosure was investigated. One derma roller had a needle length of 0.5 mm and one derma roller had a needle length of 1.5 mm.

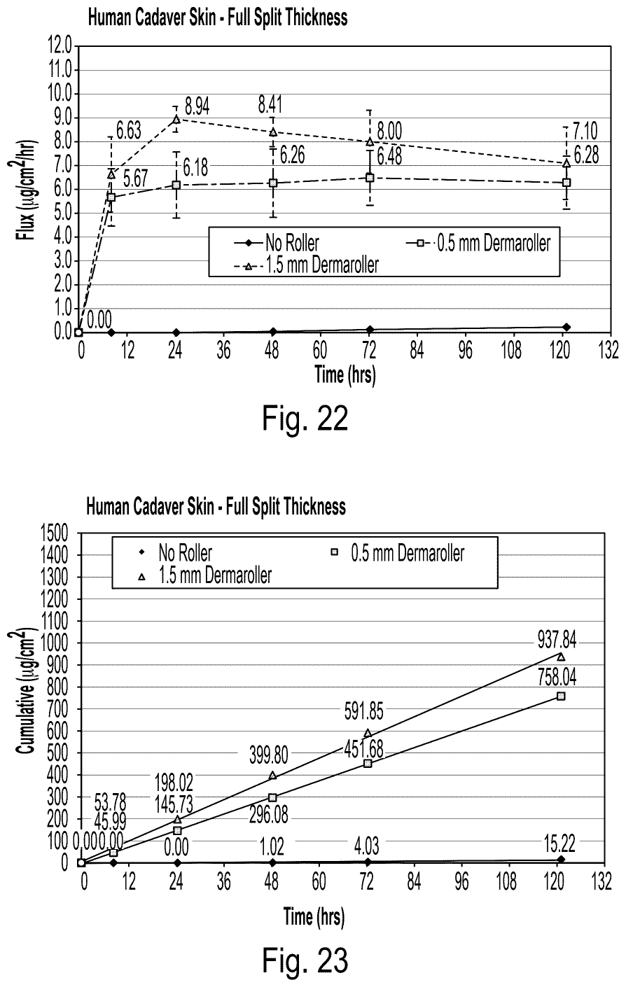

As shown in FIGS. 22-23, microneedling a surface of skin prior to application of the transdermal drug delivery systems of the present disclosure on the surface of skin results in significantly improved average flux and cumulative permeation. Further, although both derma rollers improved flux and permeation, microneedling with the derma roller with the 1.5 mm needle length improved flux and permeation to a greater extent than microneedling with the derma roller having a 0.5 mm needle length.

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A transdermal drug delivery system comprising:
a molecular solid suspension of a drug in adhesive layer including an active pharmaceutical ingredient having a water solubility of less than about 10 milligrams per milliliter and a melting point greater than about 120° C., an adhesive polymer, an insoluble carrier excipient, and at least one nonionic surfactant, wherein a weight ratio of an amount of the active pharmaceutical ingredient suspended in the molecular solid suspension of the drug in adhesive layer to the amount of the active pharmaceutical ingredient solubilized in the drug-in-adhesive layer is from about 1.2:1 to about 1000:1.

2. The transdermal drug delivery system of claim 1, wherein the active pharmaceutical ingredient has a log P value ranging from about −2 to about 8.

3. The transdermal drug delivery system of claim 1, wherein a weight ratio of the active pharmaceutical ingredient to the insoluble carrier excipient ranges from about 1:0.4 to about 1:5.

4. The transdermal drug delivery system of claim 1, wherein the insoluble carrier excipient comprises crosslinked polyvinylpyrrolidone, silicon dioxide, calcium silicate, or a combination thereof.

5. The transdermal drug delivery system of claim 1, wherein the active pharmaceutical ingredient is an immunomodulatory (IMiD) agent, a steroid, a hormone, or a central nervous system (CNS) agent.

6. The transdermal drug delivery system of claim 1, wherein the adhesive polymer comprises an acrylate copolymer, an ethylene-vinyl acetate copolymer, a vinyl acetate-acrylic copolymer, a rubber co-polymer, a polyisobutylene polymer, a silicone polymer, or a combination thereof.

7. The transdermal drug delivery system of claim 1, further comprising a humectant, wherein the humectant comprises a sugar, a sugar alcohol, a sugar ester, a polyol, phytantriol, pantothenic acid, urea, tocopherol polyethylene glycol succinate, a polyethylene glycol, hyaluronic acid, salicylic acid or derivative thereof, an alpha hydroxy acid or derivative thereof, an amino acid or derivative thereof, panthenol, or a combination thereof.

8. The transdermal drug delivery system of claim 1, wherein the surfactant comprises a first nonionic surfactant with a hydrophilic to lipophilic balance (HLB) value of less than about 12.

9. The transdermal drug delivery system of claim 8, wherein the first nonionic surfactant comprises an ethoxylated alcohol.

10. The transdermal drug delivery system of claim 9, wherein the first nonionic surfactant is Steareth-2, Oleth-2, Ceteth-3, Oleth-3, C12-13 Pareth-3, Oleth-5, C12-13 Pareth-4, Laureth-4, Ceteareth-6, Oleth-10, Steareth-10, or a combination thereof.

11. The transdermal drug delivery system of claim 8, wherein the nonionic surfactant further comprises a second nonionic surfactant with a hydrophilic to lipophilic balance (HLB) value of greater than about 18.

12. The transdermal drug delivery system of claim 11, wherein the second nonionic surfactant comprises a co-polymer of poly(ethylene oxide) and poly(propylene oxide).

13. The transdermal drug delivery system of claim 12, wherein the second nonionic surfactant is a poloxamer.

14. The transdermal drug delivery system of claim 13, wherein the second nonionic surfactant is Poloxamer 181, Poloxamer 188, Poloxamer 338, Poloxamer 407, or a combination thereof.

15. The transdermal drug delivery system of claim 1, further comprising:
an occlusive backing layer, wherein the occlusive backing layer forms an exterior facing-surface of the transdermal drug delivery system; and a release liner, wherein the release liner is positioned adjacent a skin contacting surface of the molecular solid suspension drug in adhesive layer.

16. A method of forming a molecular solid suspension of a drug in adhesive layer for a transdermal drug delivery system, the method comprising:
solubilizing an active pharmaceutical ingredient in a first process solvent to form a solution, wherein the first process solvent comprises a polar aprotic solvent;
adding an insoluble carrier excipient to a second process solvent to form a dispersion of particles, wherein the second process solvent is different from the first process solvent;
combining the solution with the dispersion of particles to form a formulation;
adding a surfactant to the formulation; and
adding an adhesive polymer to the formulation, wherein a weight ratio of an amount of the active pharmaceutical ingredient suspended in the molecular solid suspension of the drug in adhesive layer to an amount of the active pharmaceutical ingredient solubilized in the drug in adhesive layer is from about 1.2:1 to about 1000:1.

17. The method of claim 16, further comprising coating the formulation onto one of a backing layer or a release liner.

18. The method of claim 17, further comprising evaporating the first process solvent and the second process solvent.

19. The method of claim 16, further comprising applying the other of the backing layer or the release liner onto an exposed surface of the formulation.

20. The method of claim 16, wherein the active pharmaceutical ingredient has a water solubility of less than about 10 milligrams per milliliter, a melting point greater than about 120° C., and a log P value ranging from about −2 to about 8.

21. The method of claim 16, wherein a weight ratio of the active pharmaceutical ingredient to the insoluble carrier excipient ranges from about 1:0.4 to about 1:5.

22. The method of claim 16, wherein the insoluble carrier excipient comprises crosslinked polyvinylpyrrolidone or silicon dioxide or calcium silicate.

23. The method of claim 16, wherein the active pharmaceutical ingredient is an immunomodulatory agent, a steroid, a hormone, or a central nervous system (CNS) agent.

24. The method of claim 16, wherein the adhesive polymer comprises an acrylate copolymer, a polyisobutylene, a silicone, or a combination thereof.

25. The method of claim 16, wherein the surfactant comprises a first nonionic surfactant having a hydrophilic to lipophilic balance (HLB) value of less than about 12.

26. The method of claim 25, wherein the surfactant comprises a second nonionic surfactant having a hydrophilic to lipophilic balance (HLB) value of greater than about 18.

27. The method of claim 16, further comprising adding a humectant, wherein the humectant comprises a sugar, a sugar alcohol, a sugar ester, a polyol, phytantriol, pantothenic acid, urea, tocopherol polyethylene glycol succinate, a polyethylene glycol, hyaluronic acid, salicylic acid or derivative thereof, an alpha hydroxy acid or derivative thereof, an amino acid or derivative thereof, panthenol, or a combination thereof.

28. A method of delivering an active pharmaceutical ingredient to a wearer via a transdermal drug delivery system, wherein the transdermal drug delivery system comprises a molecular solid suspension of a drug in adhesive layer that includes the active pharmaceutical ingredient, wherein the active pharmaceutical ingredient has a water solubility of less than about 10 milligrams per milliliter and a melting point greater than about 120° C., an adhesive polymer, an insoluble carrier excipient, and at least one nonionic surfactant, wherein a weight ratio of an amount of the active pharmaceutical ingredient suspended in the molecular solid suspension of the drug in adhesive layer to the amount of the active pharmaceutical ingredient solubilized in the drug-in-adhesive layer is from about 1.2:1 to about 1000:1, the method comprising:
microneedling a surface of skin of the wearer with a dermally-applied device;
removing a release liner on the transdermal drug delivery system to expose a skin contacting surface of the molecular solid suspension drug in adhesive layer; and
positioning the skin contacting surface of the molecular solid suspension drug in adhesive layer to the surface of skin of the wearer.
